(19) United States
(12) Patent Application Publication
(10) Pub. No.: US 2012/0330415 A1
(43) Pub. Date: Dec. 27, 2012

(54) HAPTIC DEVICES FOR INTRAOCULAR LENS

(75) Inventors: Wayne B. Callahan, Abingdon, VA (US); Paul S. Koch, Warwick, RI (US); Anna S. Hayes, Newton Centre, MA (US); Robert E. Kellan, Methuen, MA (US)

(73) Assignee: Anew Optics, Inc., Bristol, TN (US)

(21) Appl. No.: 13/361,688

(22) Filed: Jan. 30, 2012

Related U.S. Application Data
(60) Provisional application No. 61/626,473, filed on Sep. 28, 2011.
(60) Provisional application No. 61/500,203, filed on Jun. 23, 2011.

(51) Int. Cl. A61F 2/16

(52) U.S. Cl. 623/6.43

(57) ABSTRACT

A haptic for fixation to, and manufacture in conjunction with, an intraocular lens to be implanted in the natural lens capsule of the human eye is disclosed. The haptic secures the lens in an appropriate position within the natural capsule so as to provide optimal visual acuity through the aphakic lens. The haptic ends are designed to position the lens neutrally, anteriorly or posteriorly within the lens envelope. The haptic has a of an anterior retention ring and a posterior retention ring.

### REFERENCE TO RELATED APPLICATIONS

This application claims priority to U.S. Provisional Application No. 61/118,085 entitled “Haptic Devices For Intraocular Lens” and filed Nov. 26, 2008, U.S. Provisional Application No. 61/157,781 entitled “Haptic Devices For Intraocular Lens” and filed Mar. 5, 2009, U.S. Provisional Application No. 61/184,655 entitled “Haptic Devices For Intraocular Lens” and filed Jun. 5, 2009, U.S. Non-Provisional application Ser. No. 12/626,473 entitled “Haptic Devices For Intraocular Lens” and filed Nov. 25, 2010, U.S. Provisional Application No. 61/437,291 entitled Competitive Pseudophakic Accommodating Intraocular Lens” and filed Jan. 28, 2011, and U.S. Provisional Application No. 61/500,203 entitled Competitive Pseudophakic Accommodating Intraocular Lens” and filed Jun. 23, 2011, the entirety of each of which is hereby incorporated by reference.

### BACKGROUND

1. Field of the Invention

This invention is directed to haptic devices for intraocular lenses that provide increased comfort and performance to a patient. In particular, the invention is directed to haptic devices and designs, including injectors, for insertion of intraocular lenses without rolling the lenses, and to methods for performing insertion. Specifically, the invention, along with its various iterations, is designed to provide suitable degrees of focal flexibility, or accommodation, when used in conjunction with a monofocal optic, and, in certain instances, mitigate the onset of post-surgical conditions, specifically posterior capsular Opacification.

2. Description of the Background

An intraocular lens (IOL) is an implanted lens in the eye, usually replacing the existing crystalline lens because it has been clouded over by a cataract, or as a form of refractive surgery to change the eye's optical power. The whole device usually comprises a small plastic lens with plastic side struts, called haptics, to hold the lens in place within the capsular bag inside the eye. Haptics also form the means of attachment of lenses to other areas of the eye, including the anterior angle or sulcus, the iris, and the posterior chamber ciliary sulcus. IOLs were traditionally made of an inflexible material (e.g. PMMA) though this largely been superseded by the use of flexible materials. Most IOLs fitted today are fixed monofocal lenses matched to distance vision. However, other types are available, such as multifocal IOLs which provide the patient with multiple-focused vision at far and reading distance, toric IOLs to correct for astigmatisms, and adaptive IOLs which provide the patient with limited visual accommodation.

Intraocular lenses have been used since 1999 for correcting larger errors in myopic (near-sighted), hyperopic (far-sighted), and astigmatic eyes. This type of IOL is also called PIOL (phakic intraocular lens), and the crystalline lens is not removed. More commonly, aphakic IOLs (that is, not PIOLs) are now used for visual correction errors (especially substantial hyperopia), and implanted via Clear Lens Extraction and Replacement (CLEAR) surgery. During CLEAR, the crystalline lens is extracted and an IOL replaces it in a process that is very similar to cataract surgery: both involve lens replacement, local anesthesia, both last approximately 30 minutes, and both require making a small incision in the eye for lens insertion. Patients recover from CLEAR surgery 1-7 days after the operation. During this time, patients should avoid strenuous exercise or any activity that significantly raises blood pressure. Patients should also visit their ophthalmologists regularly for several months so as to monitor the IOL implants. CLEAR has a 90% success rate (risks include wound leakage, infection, inflammation, and astigmatism). CLEAR can only be performed on patients ages 40 and older. This is to ensure that eye growth, which disrupts IOL lenses, will not occur post-surgery.

Once implanted, IOL lenses have three major benefits. First, they are an alternative to LASIK, a form of eye surgery that does not work for people with serious vision problems. Second, effective IOL implants may eliminate the need for glasses or contact lenses post-surgery. Third, the cataract will not return, as the lens has been removed. The disadvantage is that the eye's ability to change focus (accommodate) may have been reduced or eliminated, depending on the kind of lens implanted.

While significant advances have been made in the optical quality of aphakic lenses, most lenses currently made have an overall optical thickness of one millimeter or greater at the center optical focal point (e.g. see U.S. Pat. No. 4,363,142). In the late 1990's, two patents were applied for and subsequently issued for lens optics significantly thinner than the afore-referenced lens patents (U.S. Pat. Nos. 6,096,077 and 6,224,628). Although improved, the extreme thinness of the lens manufactured in accordance with U.S. Pat. No. 6,096,077 caused some minor distortions of the optic once in the eye, while the lens manufactured in accordance with U.S. Pat. No. 6,224,628 was poured of molded silicone and did not provide the desired visual acuity.

Generally, the lens separates the aqueous humor from the vitreous body. The iris separates the region between the cornea or anterior of the eye and the lens into an anterior chamber and a posterior chamber. The lens itself is contained in a membrane known as the capsule or capsular sac. When the lens is removed from the eye, the capsule may also be removed (intracapsular extraction), or the anterior portion of the capsule may be removed with the lens leaving the posterior portion of the capsule intact (extracapsular extraction), often leaving small folds or flaps from the anterior portion of the capsule. In an intraocular implant, the artificial or prosthetic lens may be inserted in the anterior chamber, the posterior chamber, or the capsular sac. The artificial lenses are usually fixedly attached within the eye, either by stitching to the iris, or by some supporting means or arms attached to the lens; in all cases the fixation mechanisms are categorized as haptics.

Several intraocular lenses designed for implant in the anterior chamber feature haptics with feet which support the lens in order to avoid the need for clips or sutures to secure the lens to the iris. The lenses worked; however, sizing the lens to fit the eye was critical to avoid complications. The lenses were made in lengths from 11.5 mm to 14 mm in 0.5 mm increments, and the thickness of the feet was about 250 microns.

A variety of lenses has been developed that provides up to four point support for the lens. The support structures for these haptics are often linked to the lens body so that the support structure should not deflect freely of the lens body, and therefore be liable to irritate portions of the eye in contact with the support structure. A variety of shapes and geometries for the lens supporting elements, or haptics, has been disclosed and described (U.S. Pat. No. 4,254,510; U.S. Pat. No. 4,363,143; U.S. Pat. No. 4,480,340; U.S. Pat. No. 4,504,981; U.S. Pat. No. 4,536,895; U.S. Pat. No. 4,575,374; U.S. Pat. No. 4,581,033; U.S. Pat. No. 4,629,460; U.S. Pat. No. 4,676,792; U.S. Pat. No. 4,701,181; U.S. Pat. No. 4,778,464; U.S. Pat. No. 4,787,902; U.S. Pat. No. Re. 33,039; U.S. Pat. No. 4,872,876; U.S. Pat. No. 5,047,052; U.K. Patent No. 2,165,456).

Despite the advances, there remain problems with intraocular implants. For example, when an intraocular lens is inserted in the eye, an incision is made in the cornea or sclera. The incision may cause the cornea to vary in thickness, leading to an uneven surface which can cause astigmatism. The insertion of a rigid lens through the incision, even with compressible haptics, requires an incision large enough to accommodate the rigid lens (typically at least 6 mm), and carries with it the increased risk of complications, such as infection, laceration of the ocular tissues, and retinal detachment. Deformable intraocular lenses made from polymethylmethacrylate (e.g. “PMMA”), polysulfone, silicone or hydrogel may be inserted through a smaller incision, about 4 mm.

It is preferred that the intraocular lens be capable of insertion through a small incision. U.S. Pat. No. 4,451,938 shows an intraocular lens in which the lens body is made in two pieces so that each piece may be inserted through the incision separately and then joined by dowels after insertion in the eye. U.S. Pat. No. 4,769,035 discloses a foldable lens which may be inserted through an incision about 3.5 mm in length.

When the intraocular lens is inserted in the anterior chamber of the eye, the feet of the haptics, or lens supporting elements, generally lodge in the scleral sulcus, a depression anterior to the scleral spur where the iris and the ciliary muscle join the sclera in the angle of the anterior chamber. The scleral sulcus is crossed by trabecular tissue in which are located the spaces of Fontana. The anterior chamber of the eye is filled with the aqueous humor, a fluid secreted by the ciliary process, passing from the posterior chamber to the anterior chamber through the pupil, and from the angle of the anterior chamber it passes into the spaces of Fontana to the pectinate villi through which it is filtered into the venous canal of Schlemm. The lens should be positioned so the flow of fluid through the trabecular tissue is not blocked or glaucoma may result.

Since the feet of the haptics of anterior chamber lenses rest in the scleral sulcus, the flow of fluid is blocked where the feet are in contact with the trabecular tissue. It is therefore desirable to decrease the amount of surface area of the haptic foot in contact with the trabecular tissue. At the same time, the haptic feet have sufficient height to prevent adhesive tissue or synechia from growing around the feet and anchoring them to the iris or cornea. The opening of the trabecula is about 200 microns, and the haptic feet of conventional intraocular lenses are usually on the order of 175 to 200 microns, effectively blocking the openings in the trabecula wherever the feet are in contact with the tissue.

Other lenses that are situated in the posterior chamber may attach to the ciliary sulcus or be positioned in the equator of the capsular sac. In haptics with attachment to the ciliary sulcus, appropriate dimensioning is essential to ensure proper anchoring. In haptics with attachment to the capsular equator, recent science demonstrates the need for appropriate dimensioning also, as the haptic must place the lens properly in the capsule. If the haptic is too short for the capsule, the lens can dislodge or rotate in the eye, events that can require additional surgery to correct and can also cause intraocular trauma. Additionally, haptics that are too short for the capsule do not allow the lens to provide the patient with any desired or designed focal flexibility (that is, accommodation). If the haptic is too long for the capsule, the lens can angle either posteriorly or anteriorly at a greater angle than designed, in the former case significantly reducing visual acuity at distance and risking reverse accommodation, in the latter case putting pressure on the iris and diminishing focal flexibility.

U.S. Pat. Nos. 5,258,025 and 5,480,428 describe a lens surrounded by a sheet-like “positioner” having projections called “supporting elements either at the four corners of or continuously around the positioner, the supporting elements being 0.3 mm long and 0.01 to 0.05 mm thick (7″a and 7″b of FIG. 3 of the '025 patent, 18 of the '428 patent). However, the lens is for implantation in the posterior chamber, the lens of the '428 actually having a length short enough to “float.” In addition, the sheet-like nature of the positioner prevents independent deflection of the feet in response to forces applied by the eye.

In addition, the lens may place a greater or lesser degree of force on the haptic feet as the lens is compressed, depending upon construction of the lens. Since the amount of pressure for a given surface area is proportional to the force, it is desirable to decrease or distribute the amount of force placed on the haptic feet in order to diminish the force applied by the feet on the trabecular tissue. This goal is achieved by mounting the haptic arms on the ends of a flexible support bar in cantilever fashion, the support bar being offset from the lens body by a stem.

The act of surgically removing the natural lens and replacing it with an intraocular lens of whatever design gives rise to certain other possible conditions that can have a profound impact on the patient's ability to see clearly over a protracted period of time, the extent of focal accommodation that can be provided to the patient, and the effective positioning of the replacement lens in the eye. These conditions normally occur in a majority of cases but may be able to be mitigated with inventive lens and haptic designs. In particular, ophthalmologists have observed that the lens capsule will tend to atrophy over time. This is in part attributable to the fact that the replacement lens rarely occupies the entire lens capsule, and most lenses tend to flatten out the capsule, thus allowing the anterior and posterior surfaces of the capsule to adhere together, causing capsular atrophy, hardening, and adhesions. All these will necessarily diminish the effectiveness of any lens claiming to offer focal accommodation. It is possible that increased circulation of the aqueous humor can preserve the suppleness of the natural lens capsule, and preventing contact between the capsular surfaces should prevent capsular adhesions.

Some physicians have advocated the use of capsular retention rings to prevent capsular atrophy. However, these rings, which are situated in the lens equator and generally used only during the surgical procedure, do not allow the ciliary body to influence the dimensions of the lens so as to provide for focal accommodation. Thus, whereas capsular retention rings may be effective when used in conjunction with non-accommodating lenses, their value with premium lenses that claim accommodation is questionable.

In some cases post surgical adhesions can occur between the lens capsule and the haptic of the intraocular replacement lens. If significant enough, these adhesions can diminish the focal accommodative functions of the lens.

Posterior Capsule Opacification (PCO) is a condition that occurs in approximately 50% of cataract patients within three years after surgery. PCO is caused by the natural migration of epithelial cells from the anterior lens capsule to the equator, thence to the posterior surface. Once the epithelial cells reach the equator, the cells die off leaving proteins that accumulate on the posterior capsular surface in the form of Elschnig's pearls or of fibroblasts that a there to the capsule and can cause significant fibroblasts, shrinkage, and clouding of the lens. If the PCO migrates to the optical area of the capsule, vision is significantly impaired. The occurrence of PCO can be mitigated surgically by means of Nd-YAG-Laser correction, which perforates the posterior capsule with small holes that deter the PCO from further migration. However, Nd-YAG laser capsulotomy surgery also carries risks of post-surgical complications including possible incursion of the vitreous into the capsule, and, as such, should be avoided if possible.

In the case of the inventive haptic designs incorporated herein, the inventors believe that the onset of PCO may be delayed or eliminated altogether through the use of appropriate haptic design to deter epithelial cell migration. In particular, 1) the design of an ultra-thin fixation plate and its appropriate sizing to fit securely at the capsular equator is intended to arrest epithelial cell migration at the haptic attachment zone and mitigate PCO accordingly, 2) a haptic design that keeps the capsule open and prevents contact between the anterior and posterior surfaces may assist in mitigating PCO onset by maintaining hydration of the capsule, 3) the quality of the cataract or CLEAR surgery can assist in retarding PCO through assiduous cleaning and polishing of the anterior capsule, and 4) the positioning of certain retention rings in the capsule, whether at the capsular equator, against the surface of the anterior capsule, and/or against the surface of the posterior capsule may arrest the migration of epithelial cells and prevent their aggregation in the posterior capsular optic zone. In some cases, IOL designers have found some success at mitigating the onset of PCO by configuring the posterior surface of the lens so as to provide a right angle at the junction of the lens with the posterior capsule. This configuration is particularly applicable for those lenses that rest entirely against the posterior capsule and do not accommodate. In other cases, IOL designers have determined that the surface quality of the haptic may have some influence on PCO mitigation.

### SUMMARY OF THE INVENTION

The present invention overcomes the problems and disadvantages associated with current strategies and designs and provides new haptic devices and methods for positioning an intraocular lens in the eye, as well as designs for specific functionality to provide optimal focal flexibility and mitigate common post surgical problems.

One embodiment of the invention is directed to haptic devices that attach to the side of an edge of a lens and at a distance from the center of the lens. Preferably the haptic has a first haptic contact point that breaks the plane of a line passing through the center of the lens at about preferably 60 degrees from preferably the twelve o'clock position of the lens and a second haptic contact point that breaks the plane of a line passing through the center of the lens at about preferably 300 degrees from the twelve o'clock position of the lens. Preferably, the haptic arm center line is an extension of the planes passing through the lens at 60 and 300 degrees and extends to intersect a circle where the center is the center of the lens and the radius is greater than the radius of the lens. Also preferably, a radially distant end connects to an arm that intersects the outside diameter of the haptic at an offset point parallel to the 12 o'clock plane of the lens.

Preferably, the haptic is designed to affix to the lens on each side of the optic edge at a sixty degree angle from the center meridian of the lens. The haptic arm is a band of the haptic material that extends outward from the optical connection then curves back inward to connect with a solid arc of haptic material concentric with the optical edge of the lens and at a distance from such optical edge to provide for a kidney-shaped open section between the lens and such haptic material. The material of the haptic is preferably flexible, thus the haptic design provides for greater thickness of the haptic in the anterior/posterior plane so as to allow for suitable positioning of the lens in the eye without anterior-posterior dislocation. The ends of the haptic may be solid, with the fixation portion of the haptic thinner or thicker than the band of material at the optical connection. Additionally, the design of the haptic at its fixation point to the capsule is intended to allow the anterior and posterior rim of the capsule to fixate to the haptic at such point(s), thus inhibiting the migration of epithelial cells from the anterior to the posterior capsule, thereby mitigating Posterior Capsule Opacification. In another embodiment, the arms of the haptic are modestly arched to increase focal flexibility.

Another embodiment of the invention is directed to haptic devices that are kidney shaped, wherein a portion of the haptic end is solid. Preferably, the solid portion of the end is thinner than the remaining portion of the haptic. The haptic may further comprise a notch at the 12 o'clock position radially proximal that allows for bending. The functionality of the inventive haptic is to anticipate natural post-surgical capsular atrophy while maintaining both a firm attachment of the haptic at the capsular equator and central positioning of the lens optic in the capsule.

Another embodiment of the invention is directed to haptic devices that are kidney shaped, wherein a portion of the haptic end is solid. Preferably, the solid portion of the haptic is configured so as to extend forward to meet the anterior capsule at some distance from the equator, and posteriorly to meet the posterior capsule also at some distance from the equator. The haptic may further comprise a notch at the 12 o'clock position radially proximal that allows for bending. The haptic may also comprise a series of small notches at the inner radius of the anterior and/or posterior feet to allow for flexing in natural response to motions of the ciliary body as well as natural differences in capsular size. The functionality of the inventive haptic is to mitigate the onset of natural post-surgical capsular atrophy by maintaining the capsule open at the equator. This should provide for enhanced circulation in the capsule of aqueous solution, which may maintain suitable levels of hydration to preserve capsular flexibility. This also may inhibit the tendency of the anterior and posterior capsules to adhere to each other, a common post-surgical occurrence with other haptic designs. Another functionality of the inventive haptic is to provide positioning of the haptic feet so as to respond to the natural flexing and stretching of the lens capsule in response to ciliary body actions, while maintaining both a firm attachment of the haptic to the capsule, and central positioning of the lens optic in the capsule.

Another embodiment of the invention is directed to haptic devices that have some open sections between the haptic feet and the optic and with haptic feet that comprise rings, arced anteriorly and posteriorly with respect to the plane of the lens optic, such that the anterior ring makes contact with the anterior capsule at some distance from the lens equator, and the posterior ring makes contact with the posterior capsule at some distance from the lens equator, the rings connected to each other and to the framework supporting the lens optic by means of ribbons and struts that maintain suitable spacing between the rings and provide for proper positioning of the lens within the capsule. The functionality of the anterior ring is to arrest epithelial cell migration across the anterior capsule, thus preventing these cells from maturing and arriving at the capsular equator. Another functionality of the inventive anterior ring is to respond to the changes of the ciliary body in such a manner as to enable the forward motion of the lens optic within the capsule to accommodate for near vision. The functionality of the posterior ring is to protect the posterior optic zone from PCO by maintaining a suitable barrier between any pearls or fibroblasts that may develop over time and block their incursion into the area behind the lens optic. Another functionality of the posterior ring is to capture the physical forces fo the ciliary body and work in conjunction with the anterior ring, the struts and the ribbons of the haptic to allow the lens optic to move within the capsule to adjust to the various stages of focal accommodation. Another functionality of the posterior ring, together with the anterior ring, the struts and ribbons is to maximize the natural circulation of the aqueous humor so as to preserve hydration throughout the lens capsule and the aqueous humor. This hydration may have the additional desirable effect of providing a mechanism whereby the spent and arrested epithelial cells can be fluished away by the aqueous humor and disposed of through the trabecular meshwork.

Another embodiment of the inventive haptic is a solid circle haptic into which are cut arced channels, preferably five, that extend from the anterior ring to the edge of the optic. These channels allow the optic to move in accommodation without distortion or decentralization, while the anterior and posterior haptic rings fix the lens centered in the capsule and maintain the capsule open.

Another embodiment of the invention is directed to haptic design to work with injectors for surgically injecting the lens and haptic into an eye of a patient. Preferably the patient is a mammal and more preferably the mammal is a human. The haptic to be injected is capable of being compressed to allow insertion into the eye. Preferably, an outer portion of the haptic is compressed into a pointed shape to aide travel through an injector and entry into the eye, and a flexible portion is thicker in the anterior or posterior plane and allows the haptic to flex for positioning within the eye without anterior/posterior movement. Also preferably, the top of the proximal end of the solid portion is attached to the bottom of the haptic portion creating an offset between the solid and flexible portions of the haptic. The distal end is capable of resting in the equator of the capsule when inserted into the eye that contained the natural lens and the posterior zonules of the eye rest against the capsule. Once position in the eye, the force created by the movement of the ciliary processes of the eye is capable of moving the zonules toward a prime meridian of the eye, the zonules in turn transfer force through the capsule that contained the natural lens and to the end of the solid portion of the haptic. The haptic is preferably capable of transferring force to the end of the flexible portion of the haptic, where the offset creates an upward, rotational force along the haptic, in turn advancing the lens forward within the eye. Also preferably, the top of the proximal end of the solid portion is attached to the bottom of the haptic portion creating an offset between the solid and flexible portions of the haptic. The distal end is capable of resting against the anterior surface of the capsule when inserted into the eye that contained the natural lens and the posterior end rests against the posterior surface of the capsule. Once positioned in the eye, the force created by the movement of the ciliary processes of the eye is capable of moving the zonules toward a prime meridian of the eye, the zonules in turn transfer force through the capsule that contained the natural lens and to the end of the haptic feet. The haptic is preferably capable of transferring this force through a series of struts that connect the anterior ring to the posterior ring and to the end of the flexible portion of the haptic, where the offset creates a forward, force along the haptic, in turn advancing the lens forward within the eye.

Another embodiment of the inventive haptic is to provide for a series of easements in the struts connecting the anterior and posterior haptic rings whereby the level of force exercised on the lens is commensurate with the desired degree of accommodative movement of the lens within the eye.

Another embodiment of the invention is directed to a haptic of the invention and further comprising a second haptic to be localized 180 degrees from the first haptic when inserted into the eye. Preferably the lens and the haptic are essentially in the same anterior posterior plane. When positioned in the eye, forward movement of the lens creates the ability to see near objects from a single focal plane lens. When the lens is positioned anteriorly to the distal end of the haptics, it creates a positive vault, and when positioned posteriorly to the distal end of the haptics, it creates a negative vault.

Another embodiment of the invention is directed to a haptic, wherein a portion of the haptic is flexible and arched when inserted into an eye to where the zonules of the eye transfer force against the solid portion of the haptic creating an upward force vector, which in turn would move the lens optic anteriorly. If the haptic is a plat haptic at its contact point with the lens equator, there is a reduced amount of clearance between the anterior and posterior surfaces of the natural lens capsule allowing the surfaces to grow together. In this case, the attached capsular surfaces and the edge of the haptic form an opening small enough to significantly reduce cell migration from the equatorial region of the capsule. If the haptic used has anterior and posterior rings, there is a stable amount of clearance between the anterior and the posterior surfaces of the lens capsule preventing the surfaces from growing together. The angle of the negative vault and the angle of the radius between the prime meridian and the optic edge are preferably equal. Also preferably, equal angles create a tangent between the capsule that contained the natural lens and the edge of the optic of the lens. When inserted into the eye, tangential forces use the capsule to seal the edge of the lens preventing cell migration under the lens.

Another embodiment of the invention is directed to a haptic wherein sections of the haptic are angled and connected by joints such that the optic rests posteriorly in the capsule for distance vision and the joints, flexing or stretching in response to the movement of the ciliary body, moves the lens anteriorly for near vision. Preferably, there are rings attached to the angled joints or segments that rest on the anterior and/or posterior surfaces of the capsule, maintaining a distance between such anterior and posterior surfaces thereby providing for continuous natural hydration of the capsule and circulation of the natural fluids of the aqueous humor. Also preferably, such rings arrest substantially the migration of epithelial cells on the anterior capsule and the proliferation of posterior capsular opacification in the focal range of the lens.

Another embodiment of the invention is directed to a method of securing a lens in a mammalian eye comprising removing a natural lens from a mammalian eye; and inserting a lens comprising the haptic of the invention into the mammalian eye.

Another embodiment of the invention is directed to devices, such as insertion devices, and methods of inserting a haptic into a lens envelope of a mammalian eye comprising the haptic of the invention.

Other embodiments and advantages of the invention are set forth in part in the description, which follows, and in part, may be obvious from this description, or may be learned from the practice of the invention.

### DESCRIPTION OF THE DRAWINGS

FIG. 1 illustrates a top view of a lens with a haptic.

FIG. 2 illustrates a sagittal view of a lens with a haptic.

FIG. 3 illustrates a ciliary process for distance vision.

FIG. 4 illustrates a ciliary process for near vision.

FIG. 5 illustrates a lens with an arched haptic.

FIG. 6 illustrates a thin edge that impedes posterior capsular opacification.

FIG. 7 illustrates a cross sectional area of a haptic end.

FIG. 8 illustrates an area of the connection between a haptic and a lens to be enlarged.

FIG. 9 illustrates an enlarged area of the connection between a haptic and a lens.

FIG. 10 illustrates a top view of a lens with a haptic comprising curved ribbons to form the kidney shape.

FIG. 11 illustrates a sagittal view of a curved-open haptic in the distance position.

FIG. 12 illustrates a sagittal view of a haptic with curved loops in the near accommodation position.

FIG. 13 illustrates a second iteration of a curved ribbon (open looped) haptic in the distance position.

FIG. 14 illustrates a second iteration of a curved ribbon (open looped) haptic in the near accommodation position.

FIG. 15 illustrates a third iteration of an open looped haptic in the distance position.

FIG. 16 illustrates a third iteration of an open looped haptic in the near accommodation position.

FIG. 17 illustrates a second iteration of an open loop kidney ribbon haptic.

FIG. 18 illustrates a fourth iteration of a haptic in the distance position.

FIG. 19 illustrates a fourth iteration of haptic in near position.

FIG. 20 illustrates a fifth iteration of an open looped haptic with anterior and posterior feet in the initial design specifications.

FIG. 21 illustrates a fifth iteration of an open loop haptic in the distance position.

FIG. 22 illustrates a fifth open loop haptic in the near accommodation position.

FIG. 23 illustrates an open loop haptic design (kidney haptic) with full anterior and posterior rings.

FIG. 24 illustrates a full circle haptic with arced grooves.

FIG. 25 illustrates another embodiment of the invention showing a lens design in cross section.

FIG. 26 illustrates another embodiment of the invention showing a lens design in cross section.

FIG. 27 illustrates another embodiment of the invention showing a top view of a lens design.

FIG. 28 illustrates the cutaway of FIG. 27.

FIG. 29 illustrates a lens with spiral anterior and posterior haptics.

FIG. 30 illustrates a lens with straight anterior and posterior haptics.

FIG. 31 illustrates

[symbol]

FIG. 32 depicts an Acuity C-Well intraocular lens as placed in an eye in association with ribbon haptics and a posterior retention ring.

FIG. 33 depicts an Adoptics IRAL lens as placed in an eye in association with ribbon haptics, a posterior plate and a posterior retention ring.

FIG. 34 depicts an Akkolens AKL-8 intraocular lens as placed in an eye in association with ribbon haptics and ribbon haptic extensions, a posterior plate, and both posterior and anterior retention rings.

FIG. 35 depicts an AMO/Visiogen Synchrony intraocular lens as placed in an eye in association with both posterior and anterior retention rings.

FIG. 36 depicts a HumanOptics AG Akkommodative ICU intraocular lens as placed in an eye in association with ribbon haptics, a posterior plate, and both posterior and anterior retention rings.

FIG. 37 illustrates a Morcher BioCom Fold 43E intraocular lens as placed in an eye in association with ribbon haptics, a posterior optic, and a posterior retention ring.

FIG. 38 illustrates a Lenstec Kellan Tetraflex intraocular lens as placed in an eye in association with ribbon haptics, a posterior plate, and both posterior and anterior retention rings.

FIG. 39 illustrates a Mehta Clamshell intraocular lens as placed in an eye in association with both posterior and anterior retention rings.

FIG. 40 illustrates a Staar Surgical Nanoflex Plate Haptic intraocular lens as placed in an eye in association with ribbon haptics, a posterior plate, and both posterior and anterior retention rings.

FIG. 41 illustrates a Tekia Tek Clear intraocular lens as placed in an eye in association with a posterior plate, a haptic arm, and both posterior and anterior retention rings.

FIG. 42 illustrates an Abbott Medical Optics Tecnis Multifocal intraocular lens as placed in an eye in association with a multifocal lens, a haptic pillar, and both posterior and anterior retention rings.

FIG. 43 illustrate an embodiment of the invention showing a lens in cross section wherein the uppermost haptic is comprised of the anterior ring designed to come into contact with the anterior capsule surface, thereby arresting lens epithelial cells' migration along the anterior capsule to the formix.

### DESCRIPTION OF THE INVENTION

The haptic device is used to affix an intraocular lens within the lens capsule once the natural crystalline lens has been removed surgically. The three specific design purposes of the haptic are: i) to permit the lens to be implanted in the eye by means of a special injector through an incision of less than about 3 mm; ii) to allow the lens to move within the posterior chamber of the eye in order to provide focal flexibility to the patient; and iii) to affix the lens in the equator of the lens capsule in such a way as to minimize the risk of Posterior Capsule Opacification (“PCO”), a negative consequence of lens replacement procedures that currently occurs in approximately 50% of patients within 2 to 3 years after surgery. Although intraocular lenses have been successfully implanted for several decades now, many of the haptic designs do not produce the desired results of mitigating PCO and/or facilitating focal flexibility (or the ability of the patient to adjust far to near vision and minimize the need for reading glasses).

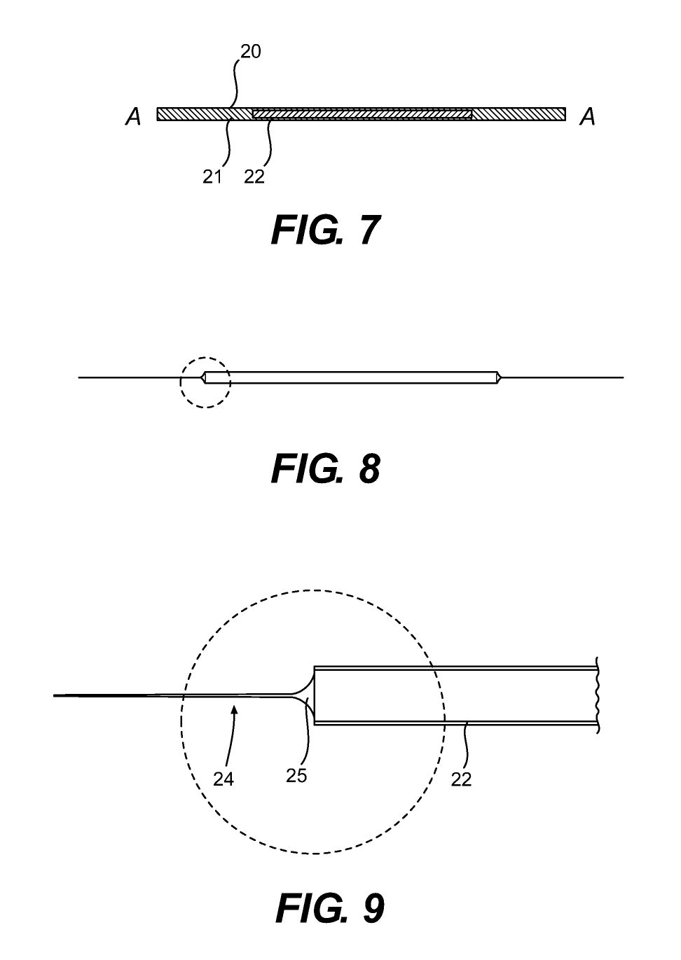

A haptic device design has been surprisingly discovered that that ameliorates PCO and facilitates focal flexibility (or the ability of the patient to adjust far to near vision and minimize the need for reading glasses). In one embodiment, the haptic of the invention is secured to the equator of the lens capsule by means of a solid but very thin plate of the same material as the attached lens, which preferably may be any of polymethylmethacrylate, hydrophobic or hydrophilic acrylate, silicone, or blends of these materials (or of the same material as the lens). The width of the plate is designed to extend beyond that portion of the lens envelope that typically closes post-removal of the natural lens (FIG. 7). Epithelial cells, normally found on the anterior surface of the inner lens capsule, can migrate to the posterior surface if their path is not impeded. A purpose of the design of the haptic of the invention is to cause a tighter closure at the edge of the haptic, which inhibits ongoing migration and growth of the epithelial cells. Moreover, the width and breadth of that portion of the haptic helps preclude migration of such epithelial cells across the anterior portion of the lens capsule to the equator. While this design may not altogether remove the risk of PCO, it retards PCO growth substantially.

A second haptic device design has been surprisingly discovered that that ameliorates PCO and facilitates focal flexibility (or the ability of the patient to adjust far to near vision and minimize the need for reading glasses). In one embodiment, the haptic of the invention is secured to the anterior capsule with an arced anterior foot, and to the posterior capsule with an arced posterior foot the effect of which is to maintain a space between the anterior capsule and the posterior capsule so as to provide for ongoing hydration of the lens capsule by the fluids of the aqueous humor. The connection between the arced anterior foot and the arced posterior foot is made by a series of struts, which may have some easements cut into them, that maintain the desired distance between the anterior and posterior feet of the haptic and optimize the accommodative force on the optic of the inventive lens, while providing for adequate fluid circulation within the capsule and the posterior chamber of the aqueous humor. Epithelial cells, normally found on the anterior surface of the inner lens capsule, can migrate to the posterior surface if their path is not impeded.

In another embodiment, a haptic design has been surprisingly discovered that has anterior and posterior haptic feet that comprise entire rings that rest on the anterior and posterior capsules, respectively, maintaining the entire capsule open and creating a barrier at both the anterior and the posterior capsular surfaces to prevent migration of epithelial cells. In this embodiment, the haptic feet are connected by a series of struts that have open spaces between, preserving the designed distance between the rings and providing for optimal fluid circulation around the inventive lens. In this embodiment also, the anterior and posterior rings may be configured so as to arrest epithelial cell migration across the anterior capsule and incursion of PCO into the optical zone of the posterior capsule, thereby providing the potential for the patient to use the intraocular lens for a substantial period of time without adverse consequences. In this embodiment, easements may be made in the struts to accommodate smaller than normal capsules, thus providing for stable concentration of the lens optic notwithstanding potential capsular size differences or changes over time. In this embodiment additionally certain easements may be made in the inner surface of the anterior and posterior rings so as to provide for responsiveness of the lens haptic to the muscular prompts of the ciliary body.

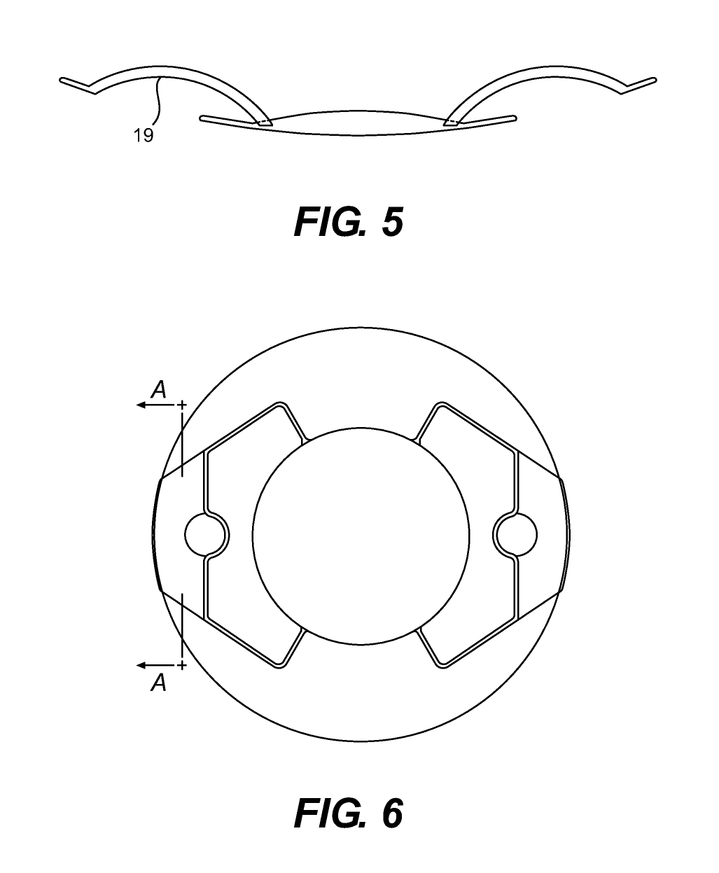

In another embodiment, the haptic of the invention may be constructed principally of a ribbon of the same material as the attached lens (as described herein). The open framework design of this portion of the haptic is to hold the optic centered vis-à-vis the retina while responding to the motion of the ciliary body so as to move the optic forward and backward in the eye, much in the same manner as a natural lens, with a minimum of lateral or oblique distortion. In the variation of this haptic design as set forth in FIG. 5, the arched portion of the haptic further facilitates the focal flexibility and causes the optic of the lens to move anteriorly as the patient focuses on near objects.

In these embodiments, the entire dimension of the lens, including both haptics and the optic, preferably varies depending upon the measurement of the natural lens capsule. The haptic has varying points of individual tailoring, including the length of the ribbon haptic (2) and (3), and the dimension of the solid end portion of the haptic. Additionally, the haptic may be used for veterinary purposes, and its overall dimensions may be increased or reduced to fit in the lens capsule of various animals.

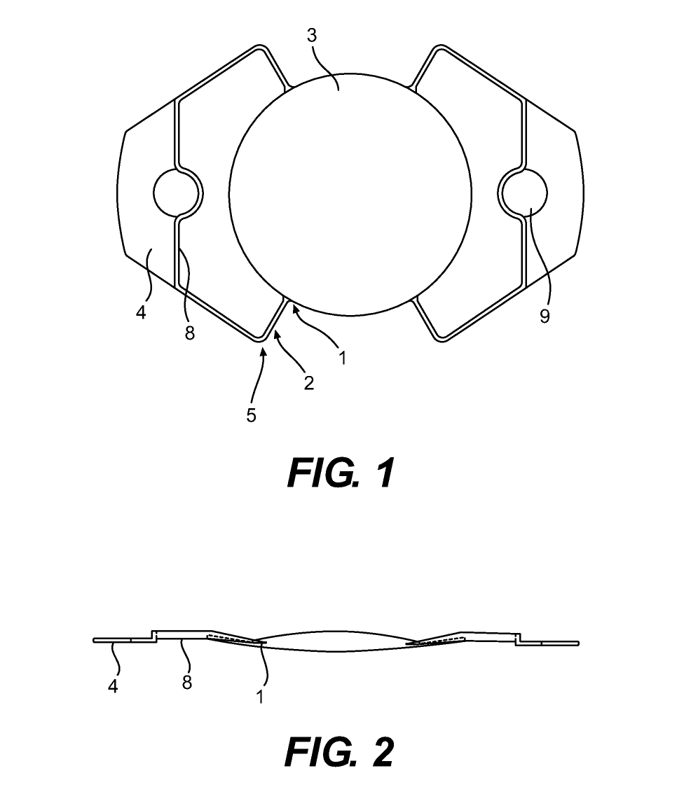

Depicted in FIG. 1 is a top view of an intraocular lens with a haptic device, and FIG. 2, a sagittal view. The haptic attachment point (1) to optic is shown along with a ribbon shaped haptic extension (2) which is in a plane through the center of the optic and the attachment point. The ribbon shaped haptic arm intersection with a circular plane larger than the radius of the optic (3). The solid end portion of the haptic (4) is parallel to a plane passing through the 12 o'clock and 6 o'clock positions of the lens. The overall shape of the haptic resembles a kidney with sharper curves (5) where the lens optic makes up a portion of the kidney. Solid end portion (4) of the haptic is thinner than the ribbon shaped sections (2).

As depicted in FIG. 1, ribbon shaped haptic (8) lies between the solid portion (4) and the end of the extended arm (2) The ribbon shaped section of the haptic (8) is shown above the solid portion of the haptic (4) in FIG. 2. Also, along the bottom, the ribbon shaped haptic (8) attaches to the solid haptic (4) along the edge (proximal to the optic) of solid portion of the haptic (4). FIG. 1 shows the notch (9) which is cut into solid haptic portion (4) to allow easy flexing for deformation into an injector.

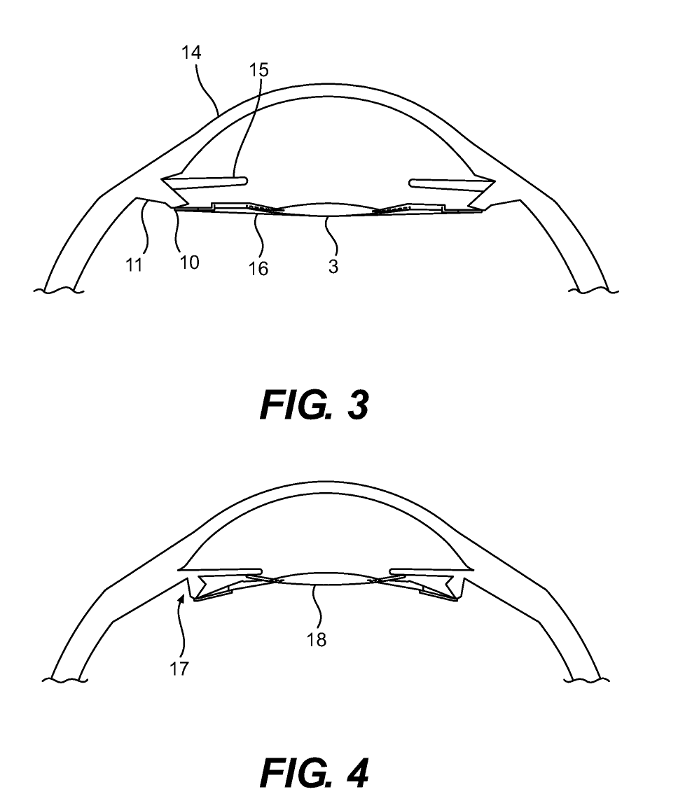

As depicted in FIG. 3, the tip (10) of the lens haptic rests against the equator of the capsule which is held in position by the zonules (11). Zonules (11) are the hair like structures that attach to the natural lens and the ciliary body and hold the natural lens in position. Zonules (11) also aide in changing the shape of the natural lens for near vision. The cornea (14) is the clear portion of the eye that refracts (bends) light. Along with the natural lens the light is bent to come to focus on the retina. The iris (colored portion) of the eye (15), also depicted in FIG. 3, is used to meter the amount of light allowed in the eye.

As depicted in FIG. 3, the intraocular lens in the far position in the eye (16), whereas in FIG. 4, the ciliary body (17) moves and changes shape to provide near vision so that the intraocular lens is in the near position (18) in the eye.

FIG. 5 depicts an arched haptic (19). As the ciliary body move force is applied to the tip of the haptic, which is transmitted into the arched haptic, which forces the haptic to compress and move anteriorly.

As depicted in FIG. 6 is one embodiment of the haptic device and optic lens, demonstrating that area of the haptic (A-A) further delineated in FIGS. 7, 8 and 9, specifically designed to mitigate PCO. FIG. 6 addresses the circular formation, described by a continuation of the indicated arc ascribed to the haptic plate, indicating an approximation of the capsular equator and the lens position within the capsule.

Depicted in FIG. 7 is a cross-sectional area of the haptic end. Shown are the anterior section of the natural lens (20), the posterior section of the natural lens (21), and the intraocular lens haptic thin solid end portion (22).

Depicted in FIG. 8 are the anterior and posterior sections of the natural lens capsule as they grow together after surgery. Depicted in FIG. 9 is an enlarged portion of FIG. 8, showing the remaining tissue (24) surrounding the solid end section (22) stretched tight. The small opening remains (25) whereby cell growth movement through the opening is impeded. With thick footplates in many cases the opening is large enough that there is little or no impediment to the cell migration; therefore, the cells deposit between the intraocular lens and the posterior capsule which opacifies the capsule and reduces the light passage.

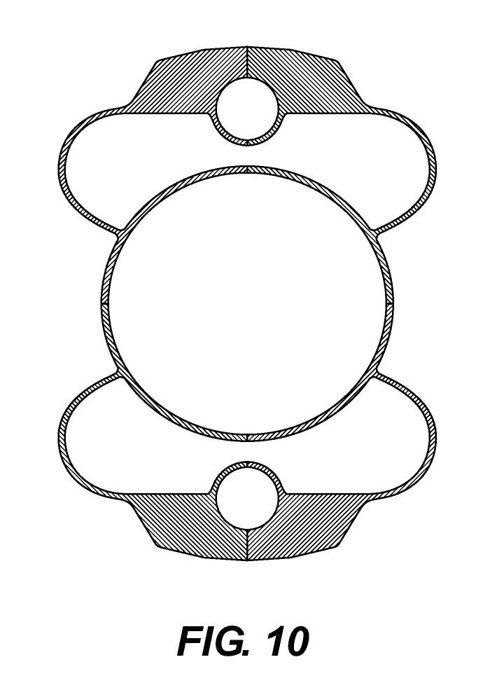

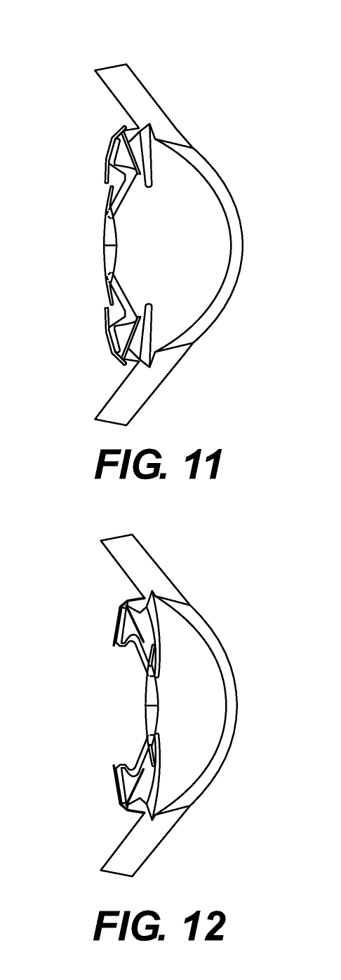

Depicted as FIG. 10 is a top view of a haptic in which the angles have been removed providing for a continuous kidney shape in which the width of the ribbon haptic is less than the depth of that haptic so as to provide for natural easement and constant centering of the lens optic while ensuring sufficient thrust strength to move the optic anteriorly and posteriorly within the capsule to provide focal accommodation. FIGS. 11 and 12 are sagittal views of such a lens haptic, demonstrating a haptic design that is configured with two angles, as in a knee and an ankle, that respond to the force of the ciliary muscles to flex and extend, thus moving the lens optic.

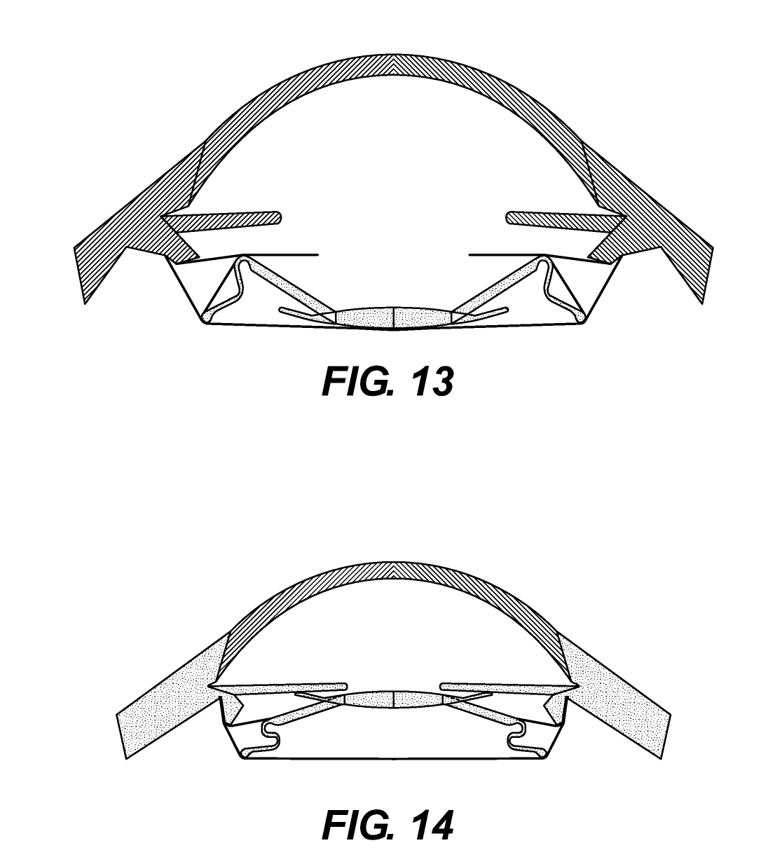

As depicted in FIGS. 13 and 14, the innovative haptic contains one or more angled or arced segments providing additional flex and thrust for moving the lens within the eye to adjust for distance and near vision. The dimensions of the angled segments may vary in accordance with the designed purpose, and may be constructed such that the width of the segments may be varied or consistent while the depth of the segments will vary according to stress calculations for that segment such that the joints of the segments flex adequately to allow the length of the segments to exert the required force on the lens optic.

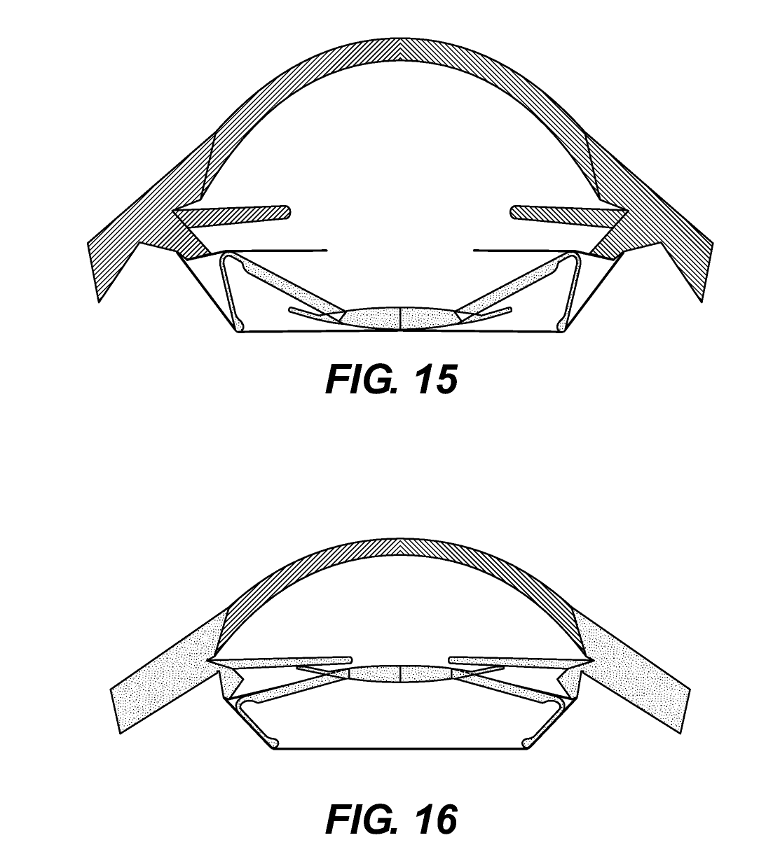

As depicted in FIGS. 15 and 16, the innovative haptic contains a knee and is designed such that the posterior foot of the haptic rests somewhat more central than the connection point of the knee with the anterior capsule.

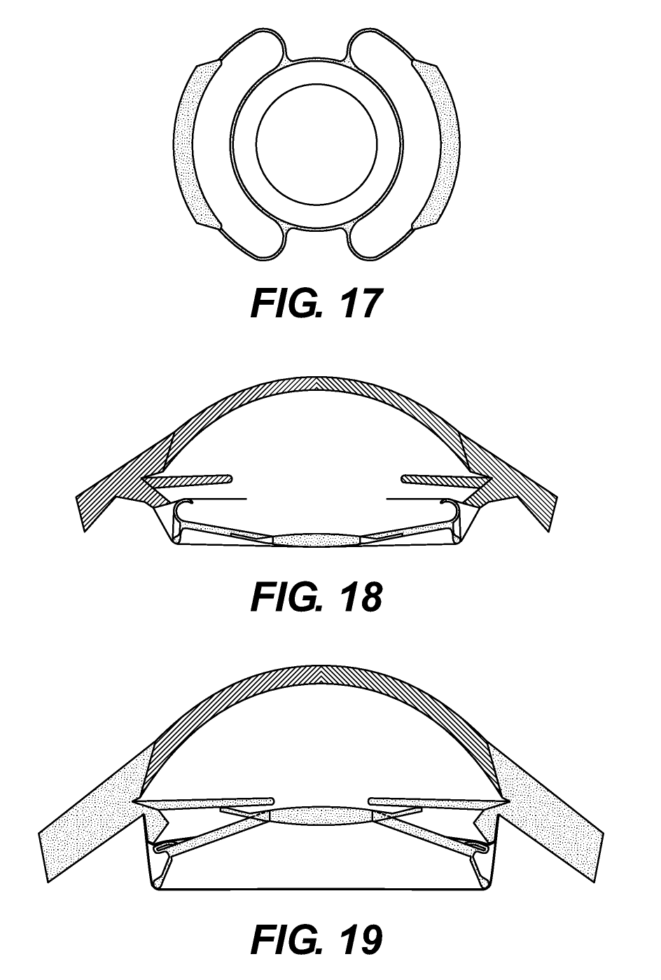

FIG. 17 illustrates a further embodiment of the kidney haptic, with FIGS. 18 and 19 demonstrating the sagittal views of such haptic, in which case the anterior haptic plate is configured to curve anteriorly toward the center of the eye. The posterior foot of the haptic rests against the posterior capsule at a certain point somewhat outside the comparable contact point of the anterior haptic, though dimensions may vary in accordance with the designed purpose of such lens.

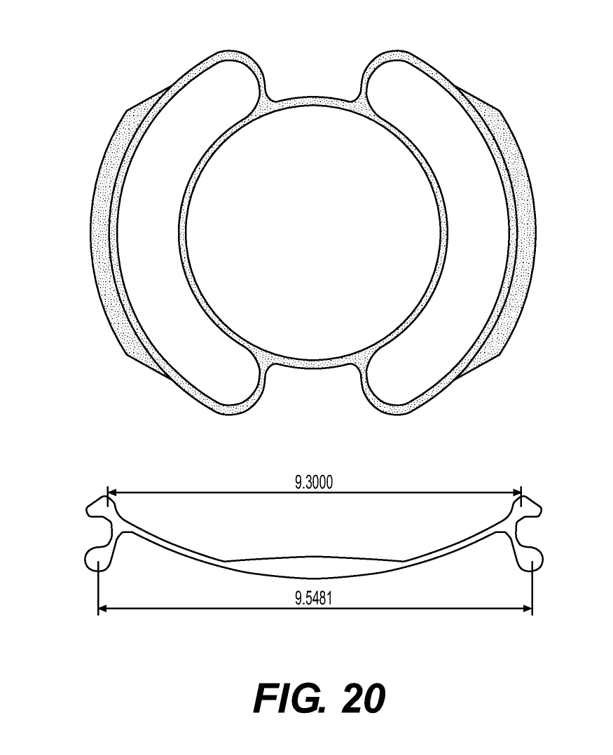

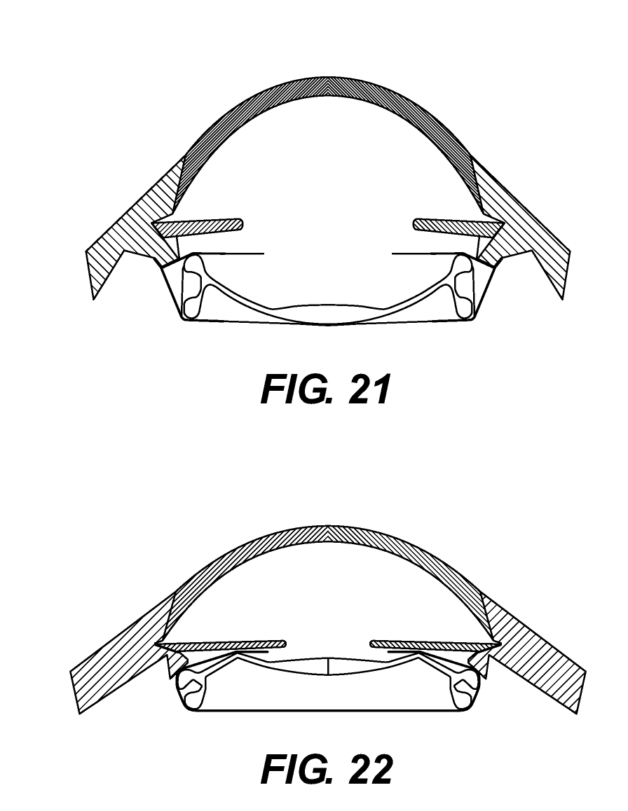

FIG. 20 depicts a further modification of the kidney haptic, showing a top view and a sagittal view with preliminary dimensions. FIGS. 21 and 22 illustrate the functionality of this inventive haptic in positions for distance and near vision.

In all of the above design manifestations, rings may also be affixed to the anterior and or posterior joints or legs of such angled segments to rest in the capsule at some distance from the equator, or with one ring in the equator and the other at some distance, to mitigate the migration of epithelial cells. In such cases the rings may contain right angles at the areas of contact with the anterior or posterior surface of the capsule. The function of such rings in conjunction with the angled segments may also be to maintain the aperture of the lens capsule distant from the equator so as to provide for continuous irrigation of the region by the normal circulation mechanisms of the aqueous humor. This may preserve the natural consistency and elasticity of the lens capsule, thus ensuring prolonged functionality of the inventive lens haptic.

Another embodiment of the invention is directed to an intra-capsular intraocular lens comprising a flexible acrylic loop that is sized to fit against the inside of the natural lens capsule across the anterior capsule, through the prime meridian or equator of the lens capsule and to a point on the posterior capsule distally outward from that central portion of the posterior capsule directly and having an optic of at least five millimeters. The haptic loop is formed as a ribbon, ideally one millimeter wide and 300 microns thick, with a natural curvature to the haptic ideally to conform to the natural curvature of the natural, crystalline lens in the accommodative, or near vision, state. One advantage of the haptic is that it maintains capsular dimension and aperture in all phases of accommodation. The haptic loop responds to the natural tension of the zonules on the lens capsule in the distant vision state, and flatten somewhat, thus exercising posterior thrust on the lens optic that is centrally suspended from the anterior haptic arms. Preferably the optic is positioned so as to be located on a plane that is anterior to the equator of the lens capsule in the accommodative state, and to be located on a plane posterior to the equator of the lens capsule in the distance vision state. The haptic responds to the relaxation of the ciliary body as it moves outward for distance vision, which increases outward tension on the zonules, thus compressing the haptic arch and moving the optic posteriorly. Conversely, as the ciliary body moves anteriorly during accommodative effort, the haptic arches reconfigure the lens capsule to a more spherical shape, with the anterior capsule of the lens in close proximity to the iris, which will move the optic anteriorly in both the lens capsule and the eye.

One function of the anterior retention ring is to arrest epithelial cells that are migrating along the anterior capsule. When these epithelial cells are removed from the capsular wall they lose their ability to adhere to any surface (i.e. once detached are not reattachable). This means that with a barrier along the anterior capsule the number of epithelial cells that arrive at the equator can be limited whence they release cortical material that can cause PCO. The posterior ring limits the extent of PCO incursion, whereas the anterior ring limits PCO creation.

The invention may also comprise a posterior haptic and optic, positioned in the eye at the same time as the anterior haptic and optic, and positioned such that the posterior haptic ribbons are placed at a right angle to the anterior haptic ribbons, thus maintaining a natural aperture and configuration of the lens capsule. The posterior haptic is preferably designed with a lens optic that extends directly from the haptic ribbons and rests securely against the posterior capsule. This optic may be structured so as to be plano, thus providing no additional optical power but serving to protect the optical area of the posterior capsule. Alternatively, the posterior optic may be engineered to provide optical power, either positive or negative, toric, refractive, or diffractive so as to enhance the accommodative effect of the anterior optic and work in harmony with the anterior optic in its accommodative response.

A preferred design of the invention comprises rings affixed on the same plane as the ribbon haptic, either anterior and posterior or both, in all cases at some distance from the natural lens equator, such that any epithelial cell migration and/or progression of PCO is arrested at the location of such rings, thus preserving a larger open optical area. These rings are preferably composed of the same material as the haptic, but may also be affixed to the lens prior to insertion into the eye.

Preferred materials for the intraocular lens comprises hydrophilic acrylic, hydrophobic acrylic, silicone or other suitable, and preferably a flexible material that is approved for intraocular use. Preferred materials retain sufficient molecular memory to provide for constant positioning of the lens against the inner capsular wall. It is also preferred that the acrylic material be flexible enough to change shape easily and respond to the prompts of the ciliary body, but resilient enough to resist cracking or other deterioration for decades. Contact and continued contact of the haptic ribbon with the lens capsule strongly hinders and even prevents migration of epithelial cells along the anterior capsule to the equator, which is the cause of Posterior Capsular Opacification (or PCO) in many post-cataract surgery patients. Preferably, the surface of the one millimeter planes of the haptic ribbon is perpendicular to the 300 micron planes such as to nestle snugly against the capsule and provide rectangular edge, which further restricts epithelial cell migration. The preferred design also maintains the open lens capsule, thus preventing the possibility of adhesions between the anterior and posterior surfaces of the capsule. Further, the open capsule also allows the aqueous humor to circulate within the capsule, which provides for enhanced hydration of the lens capsule. This enhanced hydration provides a significant advantage over models of intraocular lenses that are primarily two-dimensional in their configuration and which stretch the lens capsule out horizontally. Another advantage of the invention is that it adjusts to fit a wide variety of lens capsule sizes and shapes. All human lens capsules are not identical in circumference or volume, which means that certain intraocular lenses will not fit certain patients, and also that a lens that does fit at the time of the lens replacement surgery may cease to fit properly in the event of capsular atrophy or adhesions due to, amongst other causes, contact between the lens capsular surfaces, dehydration of certain areas of the lens capsule as a result of insufficient aqueous humor circulation, or PCO, specifically in the manifestation of Eschnig's Pearls or Soemmering's Rings. The lens of the invention, with a ribbon haptic design, preferably adjusts to fit a wide range of eyes, the limiting factor being the distance between the end points of the haptic loops on the posterior capsular surface. Moreover, the elastic pressure of the invention exerts a positive influence on the capsule, encouraging prolonged elasticity and curbing capsular contraction tendencies.

In another embodiment of the invention, preferably a second ribbon haptic mechanism is inserted in an inverse position resting against the posterior capsule, with the haptic ribbon arms extending through the capsular equator and onto the inner face of the anterior capsule. The secondary lens provides a fuller and more spherical configuration to the lens capsule, thereby providing increased damming qualities against epithelial cell migration, and maintaining the optical portion of the posterior capsule free from threats of PCO. Another aspect of the second haptic mechanism is that, in the event that the ophthalmologist determines to execute a Nd-YAG Laser Capsulotomy, the second optical piece, which may be a plano lens, serves as a permanent protection against possible prolapse of the vitreous into the lens capsule and the anterior chamber which is a potential hazard of any posterior capsulotomy.

Another embodiment of the second ribbon haptic mechanism incorporates a flexible connection between anterior and posterior haptic segments such that the anterior and posterior haptics are always fixed at 90° to each other but with sufficient flexibility to allow the haptics to move closer to or farther away from each other as the configuration of the lens capsule changes through the accommodative process. This design preserves the stability of the geometrical proportion of the two haptic structures while being as responsive as possible to the natural movement of the lens capsule through accommodation. This design provides an overall lens structure that is capable of being inserted into the eye through an incision of less than 3 millimeters, thus not requiring sutures. Also, the design provides constant and elastic support to the entire lens capsule, thus maintaining as much as possible the same configuration of the eye as existed prior to the removal of the natural, crystalline lens. This configuration provides the opportunity that the lens may be inserted in a younger patient than the normal cataract patient, using the CLEAR procedure, as preserving natural lens shape and configuration. Preferably, this design also provides an environment for a presbyopia correcting lens. Additionally, keeping the lens capsule open prolongs the useful life of the lens as the capsule remains hydrated by the aqueous humor, which prolongs and prevents the onset of capsular shrinkage and adhesions.

In another preferred embodiment, the ribbon haptics contain a series of perforations so as to increase the percentage of the lens capsule accessible to the natural hydration and circulation of the aqueous humor. A haptic ribbon may be solid structure, scored with perforations, may comprise a lattice-like structure, or any combination or variation thereof that preserves the elastic functionality of the haptic arms so as to meet the desired accommodative objectives of the lens. Preferably, design features of the haptic are particularly applicable to different types of patient, whether defined by age, race, gender, medical condition, or other criteria as a competent ophthalmologist may determine.

A preferred design of the invention incorporates an optic with a preferred diameter of 5 mm that is suspended from the anterior ribbon by means of at least two posteriorly oriented arms that extend from the outer perimeter of the ribbon and measure approximately 1.5 mm in length and up to 350 microns in width although other sizes may be used. These arms then connect to the outer edge of the optic. The length of the arms may vary as to the specific needs of the patient, the optical powers required in the accommodative process, and other factors as the ophthalmologist may determine. The optic may be configured as a spherical, aspherical, refractive, diffractive optic, such as the diopter power of the lens may require, with any blend of such optical styles as between the anterior and posterior surface of the lens. Because the optic is suspended in the center of the capsular space, the optic surface will not come into contact with the capsule at any time. By contrast, the posterior haptic ribbon connects directly to the plano optical center such that this center is in contact with the center of the posterior capsule. This mechanism protects the posterior capsule from PCO, and obviates the need for a posterior capsulotomy, thereby protecting the integrity of the lens capsule and minimizing the risk of vitreous prolapse.

In another embodiment of the invention, the optic is centrally suspended from the haptic ribbon by means of an arced segment that originates at the haptic arm at a point distally outward from the circumference of the optic and distally inward from the point at which the haptic arm contacts the prime meridian of the lens capsule. The arced segment consists of a tapered ribbon narrowest at its connection point to the optic, and preferably may or may not be hinged at the optical point of contact. The orientation of this ribbon is geometrically perpendicular to that of the haptic ribbon, with the broader expanse of the ribbon oriented anteriorly and posteriorly in the lens capsule so as to provide support for the lens movement within the capsule through the accommodative process. In another preferred embodiment the arced segments may number two or three at each connection point to the optic thereby providing for consistent centration and orientation of the lens optic at all times. In such cases, these arced segments may be solid, or may have an open work construction similar to the flying buttresses of a gothic cathedral. In another preferred embodiment, the arced segments may connect at various points along the circumference of the optic. In most any embodiment, the diameter of the optic may be increased to greater than 5.5 millimeters.

To prevent deformation of the outer portion of the optic in the accommodative process, especially when the lens optic is an ultra-thin diffractive or refractive optic, the ring may be slightly thicker and attached to and positioned immediately at the outer edge of the optic. This ring also provides a substantially sturdier connection point for the arced segments and allows for the addition of hinges to further increase motion of the optic in accommodation.

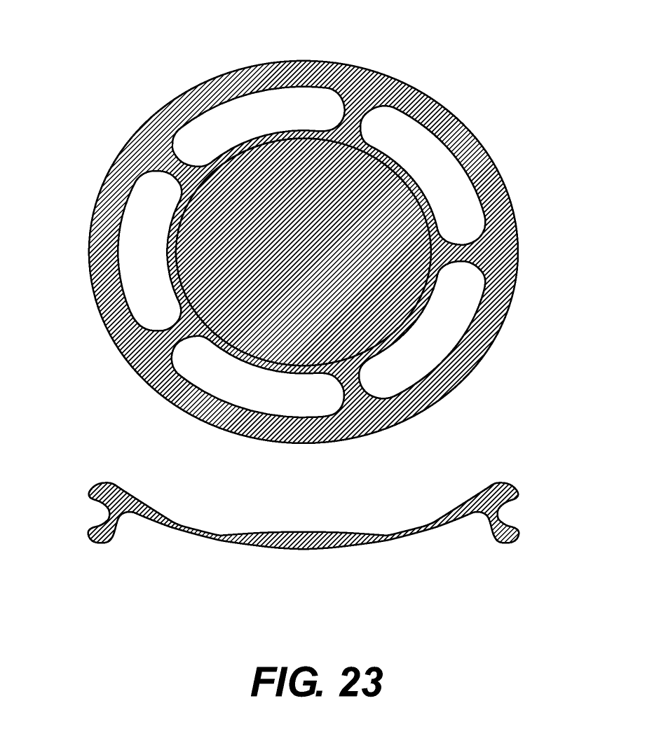

FIG. 23 depicts both top and sagittal views of the full circular haptic with ribbons and struts to create oval openings between the optic and the haptic rings. The number of contained ovals and the precise configuration of such ovals may vary according to the designed intent of the inventive haptic.

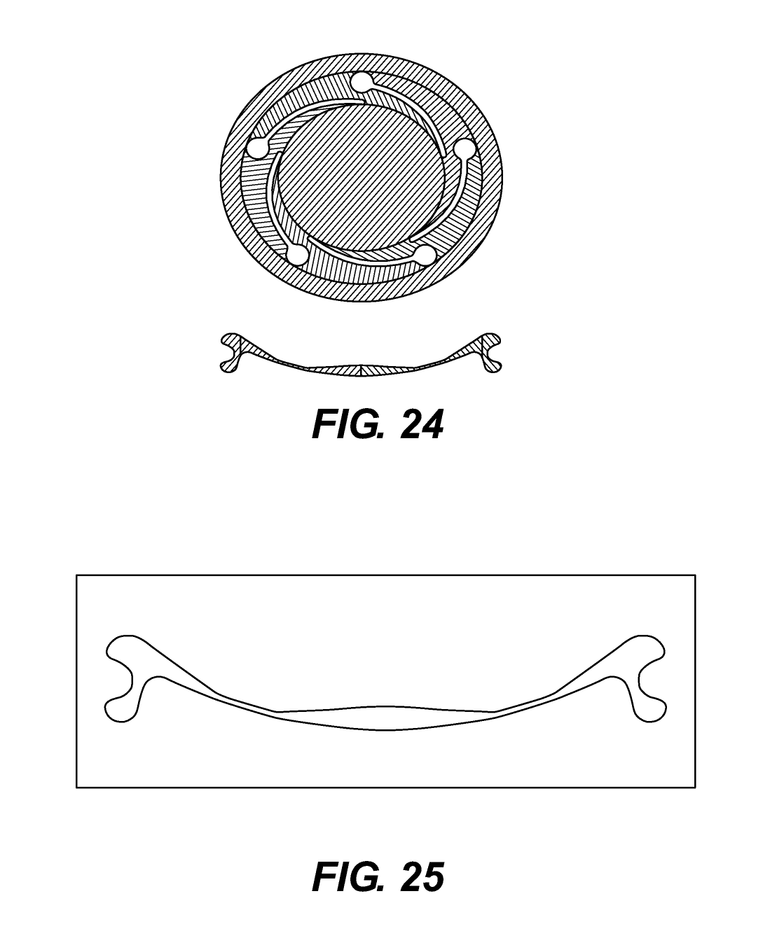

FIG. 24 depicts both top and sagittal views of a full circular haptic with arced grooves of material removed so as to provide for focal flexibility and fluid flow. In this case the number of grooves and the length and configuration of such grooves may vary in accordance with the intended purpose of the designed haptic.

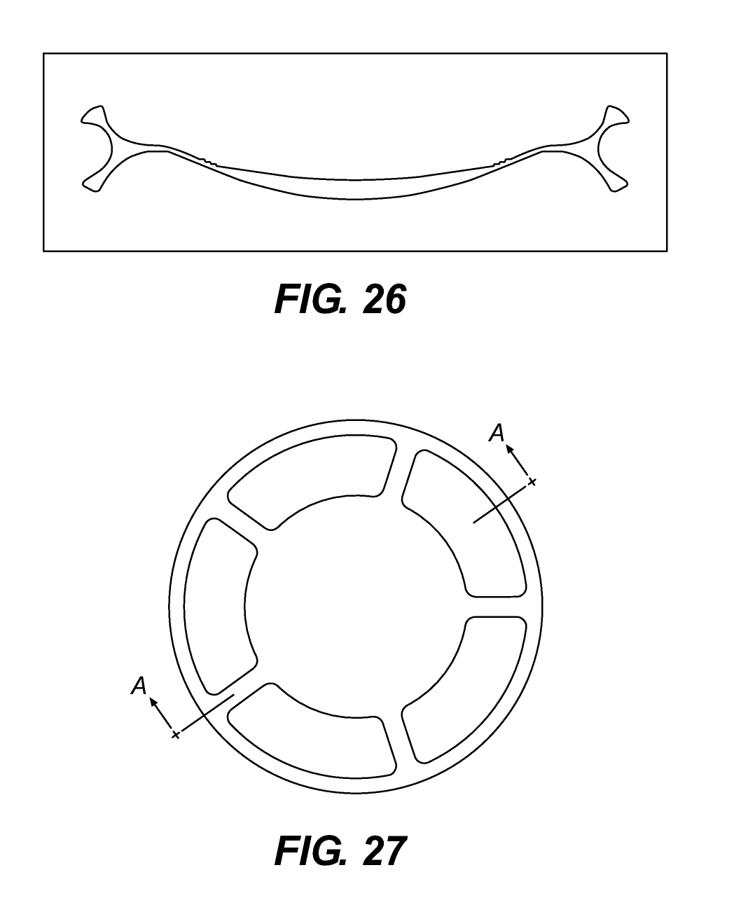

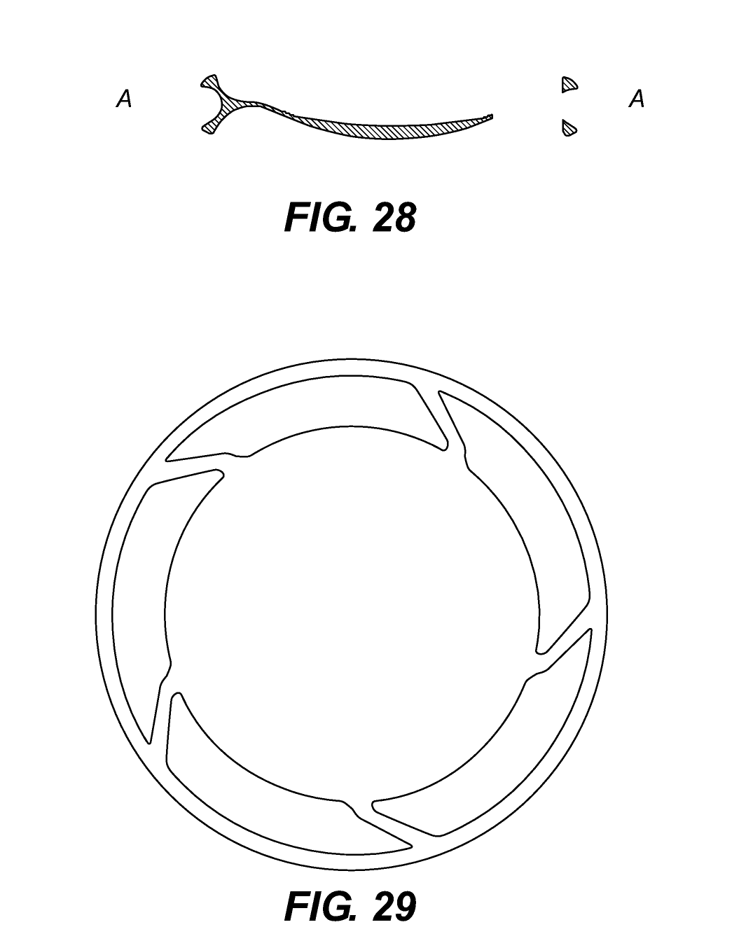

FIGS. 25 and 26 illustrate an embodiment of the lens, both shown in cross section. FIG. 27 depicts a top view of the embodiment of the lens showing full circle anterior and posterior rings. Haptic pillars connect the rings to the haptic arms and preferably only at the haptic arms. Preferably the aperture to the formix is significant and hydration occurs through the capsule. Also preferably, the anterior and/or posterior rings have modestly sharper edges at the contact points with the capsule. Preferably the lens is positioned close to the nucleus of the position of the natural lens and the center optic rests against the posterior capsule. The optic of this embodiment is preferably about 6 mm in diameter. Overall, this optic provides significant improvement to depth of field vision. FIG. 28 illustrates a transverse cross section from point A to A as shown in FIG. 27.

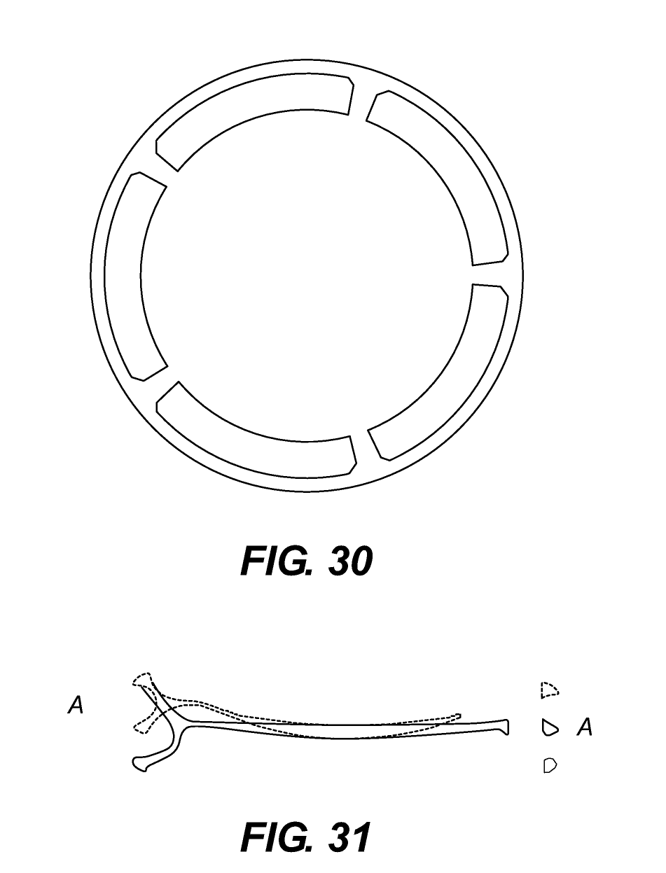

FIGS. 29-31 illustrate additional embodiments of inventive lenses. The lens depicted in FIG. 29 has spiral anterior and posterior haptics. The haptic bridges supporting the anterior and posterior rings are angled so as to provide for some posterior compression in the event of a smaller than average capsular circumference. FIGS. 30 and 31 depict a lens without the angled haptic bridge supports. FIG. 31 is a cross-section of the lens of FIG. 30. Preferably the lens has a thick anterior haptic to buttress the anterior ring, a flat anterior surface to minimize step height, and a thin bendable posterior haptic. While two shapes of haptic bridge supports are shown other haptic bridge support shapes can be used.

The following examples illustrate embodiments of the invention, but should not be viewed as limiting the scope of the invention.

### EXAMPLES

The following examples incorporate the design features and advantages of the invention into commercially available embodiments.

### Example 1

### Acuity C-Well

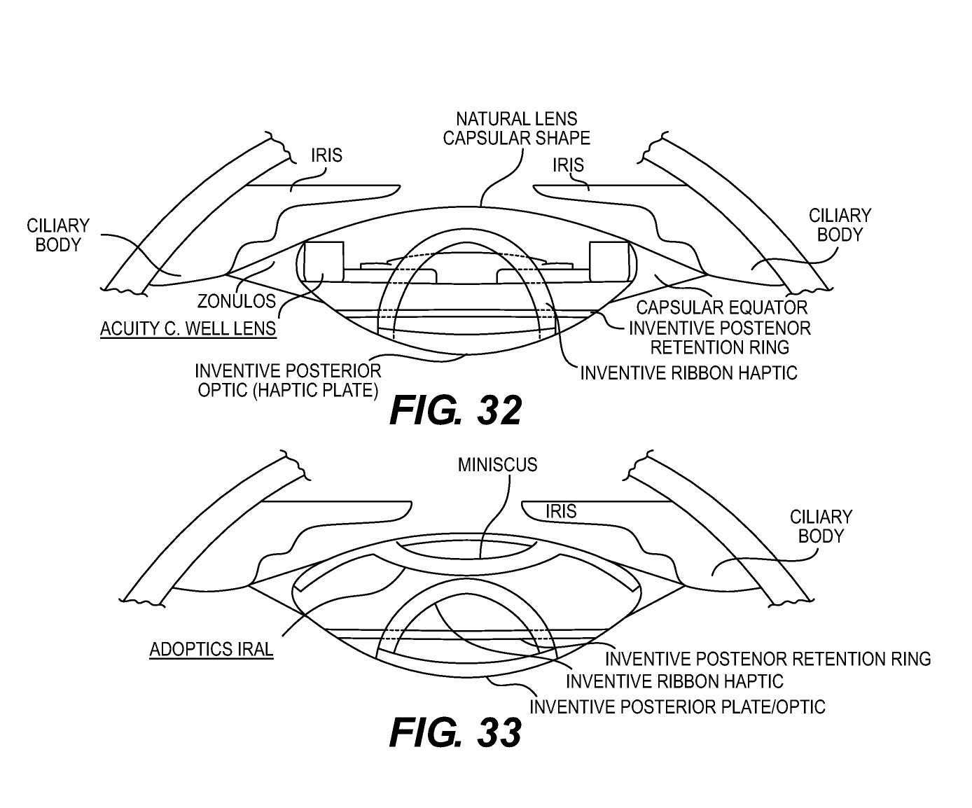

By placing a posterior haptic loop as described herein at a ninety degree angle to the axis of the extension knobs at the polar ends of the Acuity C-Well optic, the inventive loop will maintain the capsule open, allowing full circulation of the aqueous throughout the lens capsule (see FIG. 32). The optical plate of the inventive haptic may be plano, or may be given a slightly negative power so as to enhance the effective range of accommodation by providing magnification between the lenses as the C-Well optic moves forward in the eye to the accommodative state. The outer ring of the inventive haptic preferably keeps the optic area of the posterior capsule clear of any posterior capsular opacification by corralling any cortical material outside the optic range. In the event that the ophthalmologist determines to perform an Nd-YAG laser capsulotomy, the inventive haptic plate will continue to exert posterior pressure on the posterior capsule opened by the procedure, covering the hole and maintaining the vitreous in its place. This feature provides multiple benefits: while most patients who have received an ND-YAG laser capsulotomy may expect reduced performance of premium IOLs over time, the effect of the inventive haptic is to preserve the elasticity of the capsule and provide the same dynamic effect as an integral capsule; by maintaining natural positive pressure on the vitreous, the patient may have less tendency to suffer retinal detachment or macular degeneration (often consequences of the vitreous moving anteriorly in the eye); by retaining and facilitating hydration of the entire capsule, the inventive haptic should prevent adhesions, lesions, and shrinkage of the capsule, thereby enhancing the effective life of the IOL.

### Example 2

### Adoptics IRAL

The Adoptics Interfacial Refractive Accommodating Lens (IRAL) consists of an optical chamber filled with two different liquids that provides accommodation by altering the curvature of the meniscus between the lenses in response to vertical pressure at the periphery of the lens. In order for this mechanism to work, a portion of the lens consists of an anterior haptic extension that is designed to rest against the inner edge of the anterior capsule and provide the downward pressure when the ciliary body moves the lens in and forward in the eye. The rest of the lens haptic consists of two plates that rest against the equator of the lens capsule, extending it so as to effectively flatten the capsule. The inventive haptic described herein, when positioned at a right angle to the longest center line of the IRAL, combined with shortening the haptic plates of the IRAL so as not to stretch the capsule at the equator, would provide several significant benefits: 1) because the inventive haptic would keep the capsule open, this would naturally arch the IRAL anteriorly and possibly provide enhanced adjustment of the meniscus to improve the extent of accommodation; 2) keeping the capsule open would help prevent adhesion of the anterior and posterior capsule, which adhesions over time could impair the effectiveness of the IRAL, 3) mitigating the threat of posterior capsular opacification would substantially improve the long term usefulness of the IRAL, and 4) preventing vitreous prolapse or incursion into the capsule following any Nd-YAG laser procedure would also extend the useful life of the IRAL and improve patient outcomes (see FIG. 33).

### Example 3

### Akkolens AKL-8

If the posterior cube of the Akkolens AKL-8 is removed and replaced by the inventive haptic described herein, and the anterior haptic arms can be extended such as to pass through the lens equator to the posterior capsule, the posterior inventive haptic would connect to an optic with negative power, thus creating enhanced accommodative effect between the lenses. Additionally, if the anterior haptic has the addition of an anterior ring to rise modestly above the plane of the haptic such that it rests securely against the inner surface of the anterior capsule, and the inventive posterior haptic is also made with a posterior ring to rest securely against the posterior capsule, the effect would be to create a lens that not only provides superior accommodation but also blocks PCO in the optic zone (see FIG. 34).

### Example 4

### AMO/Visiogen Synchrony

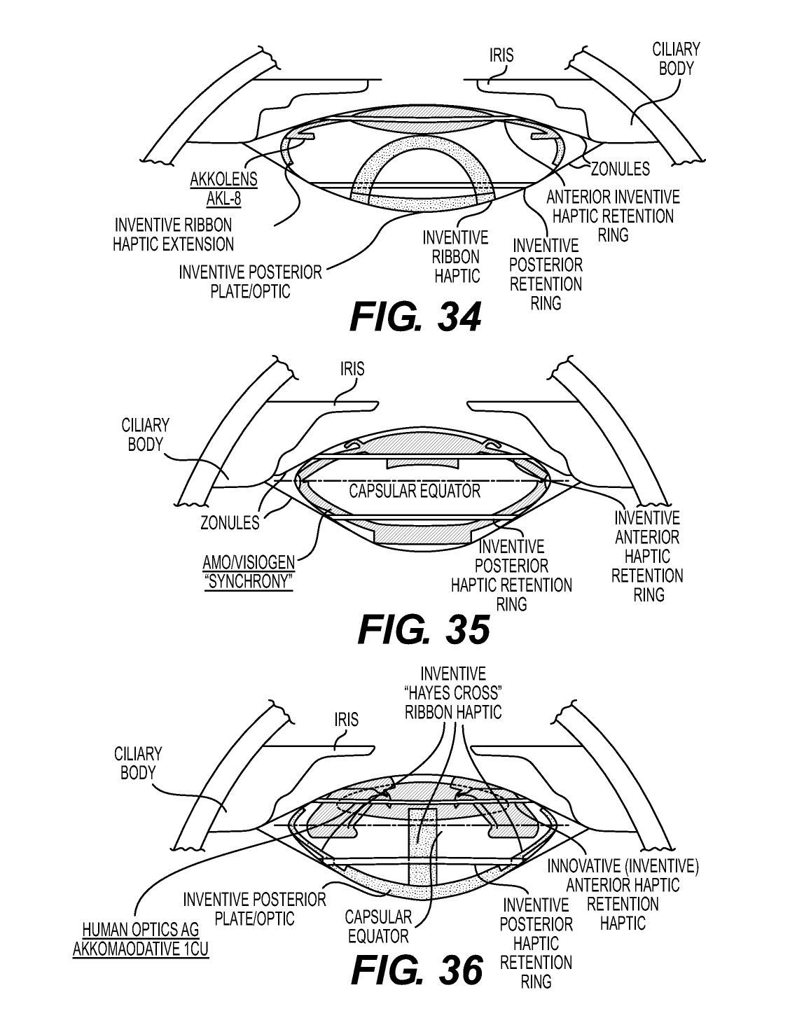

If the Synchrony lens is manufactured using a high density hydrophilic acrylic material (such as 18% or 21%) the lens could be engineered such that the haptic arms could have a maximum thickness of approximately 100 microns. Moreover, both the optic surfaces (anterior and posterior) could be made thinner. These two inventive design modifications would resolve the issue of total mass of the Synchrony, as it currently requires an incision of greater than 3.5 millimeters, thus necessitating sutures. Additionally, anterior and posterior PCO blocking rings could be located outside the optic zone on the anterior and posterior haptics to arrest epithelial cell migration and maintain the optic zone clear of cortical accumulations (see FIG. 35).

### Example 5

### HumanOptics AG Akkommodative 1CU

A modification of the inventive haptic ribbon so as to place the posterior optic centered in the optical zone of the posterior capsule with preferably four haptic arms, spaced 90° apart, either as solid or ribbon segments, extending through the equator of the capsule some distance onto the anterior capsule, and with a PCO retention ring connecting these at some distance from the optic plate, (see FIG. 36) could be placed at a 45° angle to the 1CU so as to maintain suitable expansion of the capsule to preserve hydration. The optic of the inventive haptic could be given mild negative power so as to enhance the accommodative effect of the lens. The specific benefits provided by this design modification are, among others, improved hydration of the capsule for greater irrigation and PCO mitigation, less risk of capsular adhesion anterior to posterior, or of sinecchia around the lens and haptic (the improved irrigation tending to wash away the fibrins that can cause sinecchia), improved accommodative effect due to the use of two optical surfaces, protection against vitreous prolapse or intrusion into the capsule or aqueous by providing a barrier plate, and substantially greater longevity of the lens as a result of the combination of the above benefits. A second improvement, also demonstrated in FIG. 36, is for the addition of an anterior retention ring to curb epithelial cell migration across the anterior capsule, and a posterior retention ring to curb encroachment of cortical material into the posterior capsular optic zone.

### Example 6

### Morcher BioComFold 43E

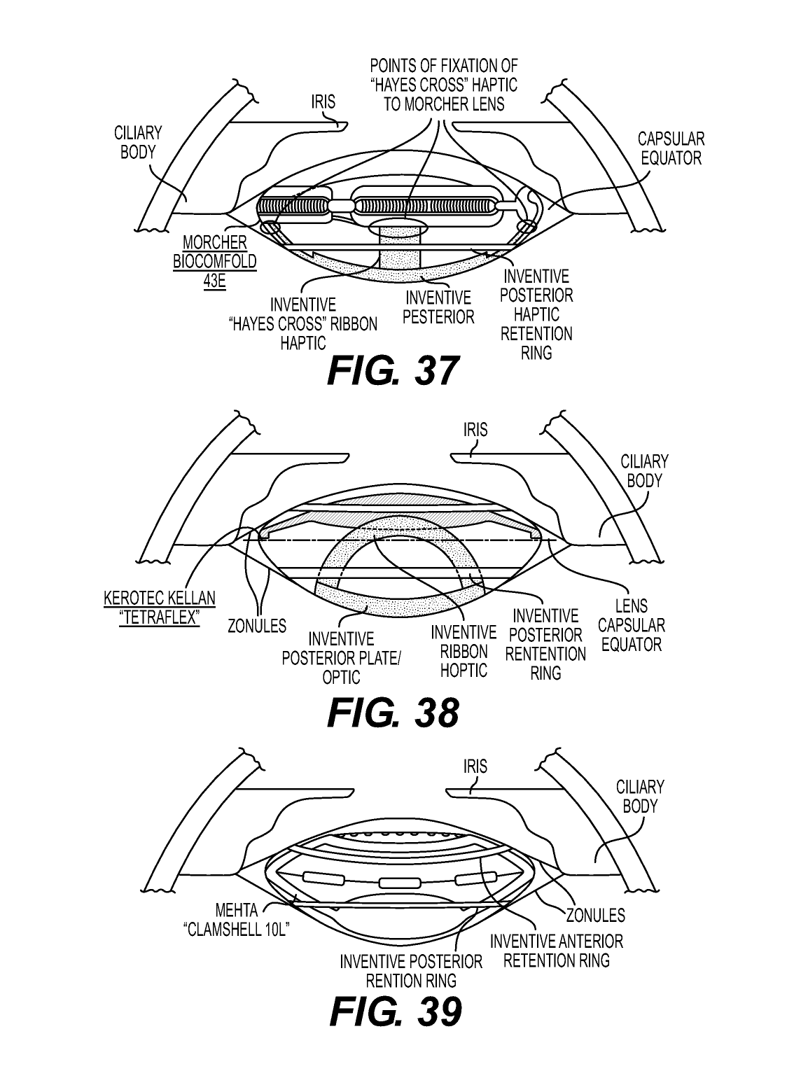

In this case the innovative modification would be to affix the inventive haptic to the posterior portion of the 43E lens haptic at four points along the lens haptic edge (see FIG. 37). The featured improvement would be an increase in accommodative power if the optical plate of the inventive haptic were configured with modest negative power. As the posterior capsule does not move as much as the anterior capsule in accommodation, the posterior haptic ring could also be affixed to the inventive haptic so as to enhance long term performance of the 43E in blocking PCO from the optic zone. Additionally, placement of the inventive haptic would maintain a greater distance between anterior and posterior capsule, thus providing the added benefits of hydration, aqueous circulation, and fibrin mitigation.

### Example 7

### Lenstec Kellan TetraFlex KH-3500

As the design of the TetraFlex provides for a 5° forward angulation in the distant vision state, pairing this lens with the inventive posterior haptic would enhance the extent of accommodation, as an open capsule would capture better the dynamics of the ciliary body and increase the forward range of motion of the TetraFlex. Additionally, by giving the posterior optic plate negative power, the range of accommodation could be further increased (see FIG. 38). The haptic design of the TetraFlex may accommodate the addition of a haptic ring rising anteriorly and slightly outward from the optic/haptic connection. In that case, to the extent that the haptic ring maintains constant contact with the anterior capsule, this ring could serve as a barrier to epithelial cell migration along the anterior capsule, thereby delaying and perhaps significantly reducing the onset of PCO. The inventive posterior haptic ring, resting securely against the posterior capsule, would effectively corral any PCO outside of the optical zone, and the optical plate, whether plano, negative and/or toric, would act as a vitreous dam in the event of an Nd-YAG laser capsulotomy. This would significantly improve the long term performance of the TetraFlex, as well as patient outcomes and satisfaction levels.

### Example 8

### Mehta Clamshell IOL

The addition of anterior and posterior rings to the Mehta Clamshell would address a key design flaw articulated by the Clamshell's inventor, Keiki Mehta MD, who acknowledged significant reduction in performance of the lens if PCO set in enough to require an Nd-YAG capsulotomy. By affixing rings to both sections of the Clamshell, on the anterior surface approximately 1.5 millimeters from the haptic equator, and on the posterior surface approximately 0.5 millimeter outside the optic, the lens, as modified, would mitigate the onset of PCO and keep the optical zone of the posterior capsule clear (see FIG. 39). Moreover, by having the rings stand proud of the rest of the Clamshell IOL, the rings would allow better circulation of the aqueous around the lens and in between the two Clamshell halves, thus providing better irrigation and flushing of the capsule. This would help prevent the proliferation of protein blasts above, beneath, and in between the IOL components.

### Example 9

### Staar Surgical NanoFlex Plate Haptic IOL

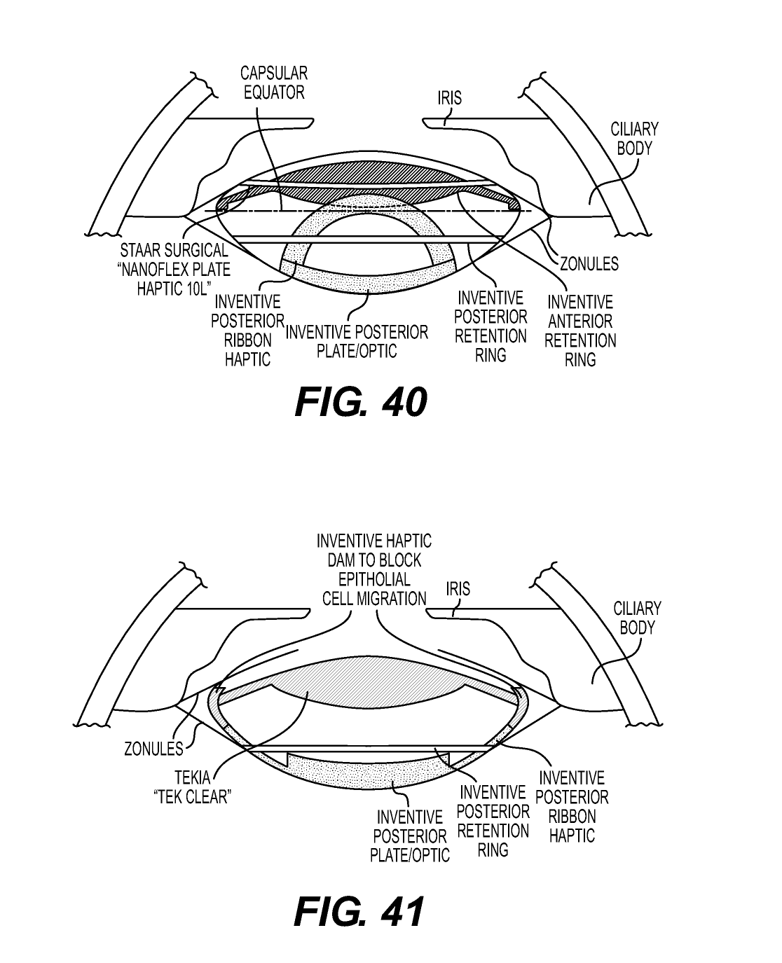

The NanoFlex is constructed using Staar Surgical's proprietary Collamer material which purports to offer superior visual acuity and range of accommodation. However, the limits of a plate haptic design, irrespective of the material of the lens and haptic, especially one such as the NanoFlex with rectangular plate haptics, are that the range of motion is necessarily limited by stretching the capsule out of its natural configuration, and that portion of the capsule not in contact with the haptic is prone to adhering to itself and shrinking, causing reduced range of motion over time. Another limiting factor of the Staar design is that it does not address PCO. The inventive haptic, placed on the posterior capsule and oriented at a 90° angle to the NanoFlex haptic, would help keep the capsule open to more of its natural shape, thus deterring the anterior and posterior capsular surfaces from making contact. This would also improve circulation of the aqueous around the lens and haptics as well as throughout the capsule, thus mitigating the risk of capsular shrinkage. By placing an anterior haptic ring between the optic and the hole cut in the plate haptic of the NanoFlex, standing above the level of the lens optic such that the ring rests securely against the anterior capsule, the NanoFlex plate haptic would only have limited contact with the anterior capsule, except for the haptic ring and the foot of the plate, thus also providing for improved irrigation of the lens and capsule on an ongoing basis (see FIG. 40). The anterior haptic ring would arrest the migration of epithelial cells along the anterior capsule, thereby delaying the development of protein strands that cause PCO. Additionally, a posterior ring on the inventive haptic would corral any PCO outside the optic zone, mitigating the need for an Nd-YAG capsulotomy, and the optic plate, whether plano, negative or toric, would dam the vitreous in the event that an Nd-YAG procedure were performed. The material in this case of the inventive lens could be Collamer or any material compatible with Collamer to produce the desired results.

### Example 10

### Tekia Tek-Clear

The Tek-Clear lens is predicated upon capturing the physical thrust dynamic of the ciliary body by placing anterior and inward pressure through the circular equatorial outer edge of the haptic on the two bridges connecting the haptic to the optic thus lifting the optic anteriorly while also altering the curvature of the anterior convex surface of the optic. This is, of course, predicated upon maintaining the structural integrity of the capsule, thus an Nd:YAG capsulotomy could impair lens performance over time. One mechanism of avoiding a posterior capsulotomy would be to impede the migration of epithelial cells along the anterior capsule, which could be achieved by modifying the lens haptic design such that at a radial distance from the center of the optic less than or equal to the distance from the center optic to the connection point of the anterior zonules to the anterior capsular surface, the haptic would be cut posteriorly so as to provide a rectangular dam to block epithelial cell migration. The effect of this modification, illustrated in FIG. 41, would be to position the lens optic and the haptic bridge somewhat posterior to the anterior capsule, thus ensuring improved aqueous circulation around the optic for LEC washing effect. The second modification to the lens would be to affix a posterior optic, connected to the Tek-Clear lens at the equator at any of two, three or more points, by means of a ribbon haptic, such that the posterior optic would rest securely against the posterior capsule so as to cover the optic zone of the posterior capsule. This would ensure against encroachment of PCO into the posterior optic zone, or, in a worst case, would protect the capsule from vitreous encroachment if an Nd:YAG capsulotomy were to be performed. The posterior haptic could be further protected from PCO by the addition of a posterior ring some distance from the outer edge of the posterior haptic and resting securely against the posterior capsule. The haptic ribbon connecting the posterior optic to the Tek-Clear equatorial haptic edge could be constructed so as to bow slightly toward the center of the capsule when the lens is in its accommodative state, preserving positive pressure against the posterior optic and enhancing accommodative effect. Moreover, the posterior optic could be configured with either modestly negative power, plano (no power), or toric adjustment so as to enhance the optical effectiveness of the lens' principal optic.

### Example 11

### Abbot Medical Optics Tecnis

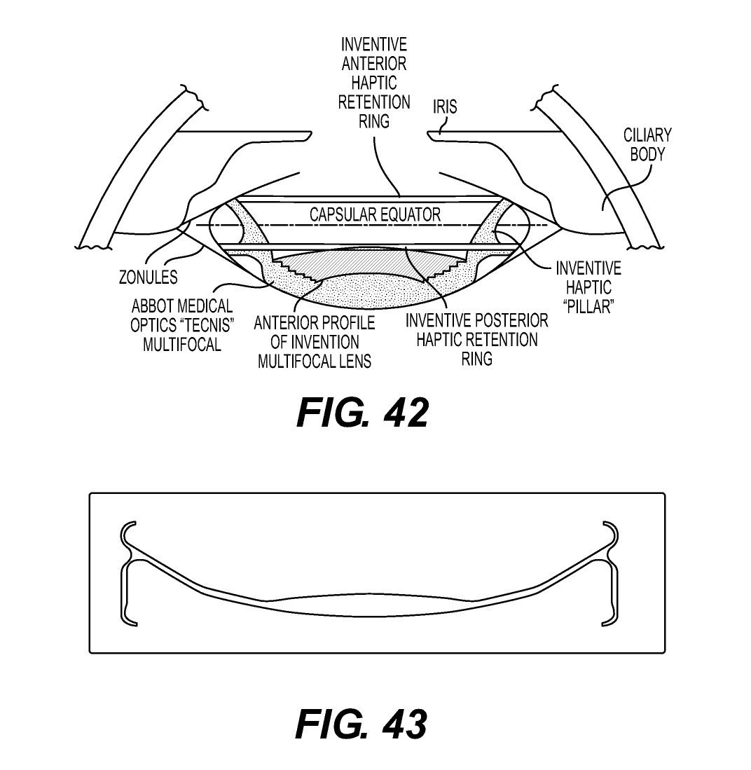

The Tecnis is a multifocal single-piece hydrophobic acrylic with a pair of modified c-loop haptics that effectively stretch out the capsule to a practically two-dimensional or flat configuration. The PCO mitigating features of the Tecnis are in the 90° angle to the optic edge, the ideal position of the optic against the central or optic zone of the posterior capsule, and the creation of a shelf, preserving the 90° angle at the haptic/optic junction. However, observation indicates that PCO incursion onto both the anterior and posterior of the optic is still a problem, and particularly at the optic/haptic junction. This would indicate that the only assurance of long term performance of the Tecnis is when the lens implantation is followed by an Nd:YAG capsulotomy. To improve upon the performance of the lens long term, and avoid a posterior capsulotomy, the multifocal optic could be affixed to the inventive haptic described herein by means of a series of haptic bridges comprising two or more ribbon bridges such that the Tecnis optic continues to be positioned against the center of the posterior capsule, but that the capsule, instead of being flattened, is kept open and the anterior and posterior haptic rings of the inventive haptic ensure that: a) epithelial cells migrating along the surface of the anterior capsule are arrested, fall away and may be washed away by the aqueous, b) the posterior haptic ring prevents encroachment of any PCO into the optical zone of the lens, and c) maintaining an open capsule should prevent eventual capsular shrinkage and the development of sinecchia or adhesions by preserving capsular hydration (see FIG. 42). In addition, using the inventive design, the mass of the optic could be reduced to mitigate the increased haptic bulk and enable small incision insertion into the capsule.

### Example 12

### All C-Loop Haptic Monofocal, Multifocal, Toric Lenses

The inventive haptic consisting of two annular haptic rings positioned at some distance from the capsular equator, in any of the modifications set forth herein, and possibly including the addition of a protective posterior optic will improve the long term performance of any and each of these lenses and the well-being of their recipients by avoiding Nd:YAG capsulotomy and ensuring hydration of the capsule while mitigating the onset of PCO.

### Example 13

### PCO Blocking Lens

FIG. 43 illustrate an inventive lens shown in cross section whose uppermost haptic is comprised of the anterior ring designed to come into contact with the anterior capsule surface, thereby arresting lens epithelial cells' migration along the anterior capsule to the formix. This anterior ring is designed with an inventive curvature such that the ring continually maintains contact with the anterior capsule in both a distance and near vision state and in all intermediate states. The lowermost portion of the inventive haptic is comprised of the posterior ring designed to maintain contact with the posterior capsule at a point distally outward of the optical zone, thereby preventing incursion of posterior capsule opacification into the optical zone of the posterior capsule. The haptic pillar connecting the anterior and posterior rings may be solid or may have apertures cut into it, in the case of the former design, to restrict any posterior capsule opacification to the area of the formix, or capsular equator, and in the case of the latter to permit hydration of the entire lens capsule by allowing circulation of the aqueous humor throughout the capsule. The inventive lens haptic suspends the optic posteriorly from a haptic that is connected to the haptic pillar and is placed posterior to the anterior haptic ring so as to prevent the lens optic from coming into contact with the anterior capsule. The haptic supporting the optic may be perforated in different patterns and at different intervals so as to allow circulation of the aqueous humor as well as permit the optic to move anteriorly and posteriorly within the capsule in response to the natural movements of the ciliary body so as to provide focal accommodation.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising, where ever used, is intended to include the terms consisting and consisting essentially of. Furthermore, the terms comprising, including, and containing are not intended to be limiting. It is intended that the specification and examples be considered exemplary only with the true scope and spirit of the invention indicated by the following claims.

1. An intraocular lens, comprising:
an optic;
a plurality of haptic arms extending from the optic;
an annular ring coupled to the plurality of arms, wherein the annular ring is comprised of an anterior retention ring and a posterior retention ring.

2. The intraocular lens of claim 1, wherein the anterior retention right and the posterior retention ring are separated by a plurality of haptic pillars.

3. The intraocular lens of claim 2, wherein each haptic pillar is coupled to one haptic arm.

4. The intraocular lens of claim 2, further comprising gaps between the haptic pillars.

5. The intraocular lens of claim 1, which is comprised of a hydrophilic acrylic, hydrophobic acrylic, silicone, or combinations thereof.

6. The intraocular lens of claim 1, which is comprised of a material for insertion into a human eye.

7. The intraocular lens of claim 1, which has a diameter greater than or equal to 9 millimeters.

8. The intraocular lens of claim 1, which has a diameter of less than or equal to 9 millimeters.

9. The intraocular lens of claim 1, which has a diameter greater than or equal to 5 millimeters.

10. The intraocular lens of claim 1, which has a diameter of less than or equal to 5 millimeters.

11. The intraocular lens of claim 1, wherein the optic has a zero optical power.

12. The intraocular lens of claim 1, wherein the optic has a negative optic power.

13. The intraocular lens of claim 1, wherein the optic has a positive power.

14. The intraocular lens of claim 1, further comprising a haptic ribbon coupling the optic to the haptic arms.

15. The intraocular lens of claim 14, wherein the haptic ribbon has one or more rounded corners.

16. The intraocular lens of claim 14, wherein the haptic ribbon has dimensions of 1 millimeter in width and 300 microns in depth.

17. The intraocular lens of claim 14, wherein the ribbon has a width of greater than or equal to 1 millimeter.

18. The intraocular lens of claim 14, wherein the ribbon has a width of less than or equal to 1 millimeter.

19. The intraocular lens of claim 14, wherein the ribbon has a depth of less than or equal to 300 microns.

20. The intraocular lens of claim 14, wherein the ribbon has a depth of greater than or equal to 300 microns.

21. The intraocular lens of claim 1, wherein the annular ring is perforated with spaces or holes to form a lattice or braid that is rectilinear, curvilinear, geometric or free-form.
